SEQUENCE LISTING (43 sequences)

SEQ ID NO: 1 - protein, 6 aa, synthetic construct
ENIYSN

SEQ ID NO: 2 - skipped

SEQ ID NO: 3 - protein, 9 aa, synthetic construct
QHFWTTPRT

SEQ ID NO: 4 - protein, 8 aa, synthetic construct
GFTFSDYA

SEQ ID NO: 5 - protein, 8 aa, synthetic construct
ITDGGSYA

SEQ ID NO: 6 - protein, 11 aa, synthetic construct
SRDRWPYYFDF

SEQ ID NO: 7 - protein, 6 aa, synthetic construct
QSIDSY

SEQ ID NO: 8 - skipped

SEQ ID NO: 9 - protein, 12 aa, synthetic construct
QCTDYSINYIGA

SEQ ID NO: 10 - protein, 8 aa, synthetic construct
GFSLSNYW

SEQ ID NO: 11 - protein, 7 aa, synthetic construct
ITSSDNT

SEQ ID NO: 12 - protein, 12 aa, synthetic construct
ARDISGINSVVL

SEQ ID NO: 13 - protein, 8 aa, synthetic construct
QSVYNDAY

SEQ ID NO: 14 - skipped

SEQ ID NO: 15 - protein, 12 aa, synthetic construct
QGAYYSNDWYYV

SEQ ID NO: 16 - protein, 7 aa, synthetic construct
GFSLSYD

SEQ ID NO: 17 - protein, 7 aa, synthetic construct
IDTSSNT

SEQ ID NO: 18 - protein, 17 aa, synthetic construct
ARGGVHAYAYAPAAFDP

SEQ ID NO: 19 - protein, 107 aa, synthetic construct
DIQMTQSPTSLSVSVGETVTITCRISENIYSNLAWYQQKEGKSPQLLVYAATKLADGVPSRFSGSGSGTQYSLKINSLQSEDFGNYYCQHFWTTPRTFGGGTKVEIK

SEQ ID NO: 20 - protein, 118 aa, synthetic construct
EVQLVESGGVLVKPGGSLKLSCAASGFTFSDYAMSWVRQTPEKRLEWVATITDGGSYAYYTDDVKGRFTVSRDNARNNLFLQMSHLRSEDTGIYYCSRDRWPYYFDFWGRGTTLTVSS

SEQ ID NO: 21 - protein, 110 aa, synthetic construct
DVVMTQTPASVSEPVGGTVTINCQASQSIDSYLSWYQQKPGQPPKFLIYGASNLASGVPSRFKGSRSGTEYTLTISDLECADAATYYCQCTDYSINYIGAFGGGTKVVVE

SEQ ID NO: 22 - protein, 116 aa, synthetic construct
QSLEESGGRLVTPGTPLTLTCKASGFSLSNYWMSWVRQAPGKGLEWIGFITSSDNTYYASWAKGRFTISKASSTTVGLKMTSPTTEDTATYFCARDISGINSVVLWGQGTLVTVSS

SEQ ID NO: 23 - protein, 112 aa, synthetic construct
APVLTQTPSPVSAAVGGTVTISCQSSQSVYNDAYLAWYQQKPGQPPKLLIYLASTLASGVPSRFSGSGSGTQFTLTISGVQCEDAATYYCQGAYYSNDWYYVFGGGTEVVVK

SEQ ID NO: 24 - protein, 122 aa, synthetic construct
QEQLEESEGGLFKPTETLTLTCTVSGFSLSYDITWVRQAPGNGLEWIGTIDTSSNTYHATWAKSRSTITKNTNLNTVTLKMTSLTAADTATYFCARGGVHAYAYAPAAFDPWGPGTLVTISS

SEQ ID NO: 25-34 - skipped (10 entries)

SEQ ID NO: 35 - protein, 107 aa, synthetic construct [sequence omitted]

SEQ ID NO: 36 - protein, 330 aa, synthetic construct [sequence omitted]

SEQ ID NO: 37 - protein, 329 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 38 - protein, 356 aa, synthetic construct
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKGNRCGQ

SEQ ID NO: 39 - protein, 107 aa, synthetic construct
PPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVK

SEQ ID NO: 40 - protein, 13 aa, synthetic construct
NAVPPVIHSPNDH

SEQ ID NO: 41 - protein, 15 aa, synthetic construct
LIPCTVYFSFLMDSR

SEQ ID NO: 42 - protein, 15 aa, synthetic construct
ITIDVTINESISHSR

SEQ ID NO: 43 - protein, 21 aa, synthetic construct
SEDLKRSYVCHARSAKGEVAK

(19) United States
(12) Patent Application Publication
(10) Pub. No.: US 2026/0317434 A1
(43) Pub. Date: Oct. 15, 2026

(54) ANTI-IL1RAP ANTIBODIES

(71) Applicant: OTSUKA PHARMACEUTICAL CO., LTD., Tokyo (JP)

(72) Inventors: Camilla Rydberg MILLRUD, Lund (SE); David LIBERG, Lund (SE); Elin Jaensson GYLLENBÄCK, Lund (SE); Kjell SJÖSTRÖM, Lund (SE)

(73) Assignee: OTSUKA PHARMACEUTICAL CO., LTD., Tokyo (JP)

(21) Appl. No.: 19/481,406

(22) PCT Filed: May 3, 2024
(86) PCT No.: PCT/EP2024/062207

§ 371 Date: Nov. 4, 2025

(30) Foreign Application Priority Data
May 5, 2023 (EP) 23171919.6

(51) Int. Cl. C07K 16/28; A61P 35/00; G01N 33/575

(52) U.S. Cl. CPC C07K 16/2866; A61P 35/00; G01N 33/5759; C07K 2317/33; C07K 2317/52; C07K 2317/565; C07K 2317/732; C07K 2317/76; C07K 2317/92; G01N 2333/7155

(57) ABSTRACT

The present invention relates to antibodies or antigen-binding fragments thereof with binding specificity for interleukin-1 receptor accessory protein (IL1RAP). Furthermore, it relates to a polynucleotide encoding said antibodies, a vector comprising said polynucleotide and a recombinant host cell comprising said polynucleotide or said vector. Further, it relates to a method for producing said antibodies or antigen-binding fragments. Further, it relates to a composition comprising said antibodies or antigen-binding fragments, said polynucleotide, said vector and/or said host cell. Further, it relates to said antibodies or antigen-binding fragments, said polynucleotide, said vector, said host cell and/or said composition for use in medicine and/or for use in the prevention and/or treatment and/or alleviation and/or detection and/or diagnosis of a disease or disorder susceptible to treatment with an inhibitor of IL-1α, IL-1β, IL-33, IL-36α, IL-36β and/or IL-36γ signaling, optionally wherein the disease or disorder is associated with cells expressing IL1RAP.

### TECHNICAL FIELD

The present invention relates to anti-IL1RAP antibodies or fragments thereof with anti-inflammatory, anti-fibrotic and/or anti-neoplastic properties, and their use in the prevention, treatment, alleviation, detection and/or diagnosis of diseases or disorders associated with IL-1α, IL-1β, IL-33, IL-36α, IL-36β and/or IL-36γ signaling.

### BACKGROUND

The interleukin-1 (IL-1) family of cytokine ligands and its receptors is associated with inflammation, autoimmunity, immune regulation, fibrosis, cell proliferation, tumour growth, tumour metastasis and host defense. It contributes to the pathology of inflammatory, autoimmune, immune regulatory, fibrotic and degenerative diseases and disorders, as well as to cell proliferative or neoplastic diseases or disorders, such as cancer (Garlanda C, The interleukin-1 family: back to the future. Immunity, 39:1003-1018 (2013)).

All these diseases cause a tremendous burden on the individual and the society and there remains a need for therapies to treat, ameliorate, prevent, diagnose or detect inflammatory, autoimmune, immune regulatory, fibrotic, degenerative, and cell proliferative diseases or disorders associated with the IL-1 family of cytokine ligands and receptors.

The IL-1 family comprises a number of agonist cytokines including IL-1α, IL-1β, IL-33, IL-36α, IL-36β and IL-36γ, and each of these cytokines bind their specific IL-1 family cell membrane receptor. Upon binding to of the cytokine to its cognate receptor, a co-receptor called IL-1 Receptor Accessory Protein (IL1RAP) is recruited to form a receptor complex that triggers intracellular signal transduction and activation of a set of transcription factors, including NF-κB, which triggers an inflammatory response. IL1RAP is the co-receptor for the IL-1 receptor I (IL1R1; binding IL-1α and IL-1β), IL-33 receptor (ST2, IL1RL1; binding IL-33), and the IL-36 receptor (IL1RL2; binding IL-36α, IL-36β and IL-36γ) and is required for signal transduction downstream of the cytokines IL-1α, IL-1β, IL-33, IL-36α, IL-36β and IL-36γ.

Antibodies binding to IL1RAP can block cytokine signaling downstream of IL1RAP-dependent receptors. Interestingly, apart from this blocking function, antibodies binding IL1RAP can also be used with regard to other functions, for example to induce antibody-dependent cell-mediated cytotoxicity (ADCC) and/or antibody-dependent cellular phagocytosis (ADCP), thereby leading to the killing of target cells, such as IL1RAP-expressing tumour cells.

In the past, antibodies have been generated which are capable of decreasing, inhibiting, and/or blocking signaling pathways of cytokine ligands of the IL-1 family, depending on the properties of the respective antibody. For example, WO 2022/136569 discloses an anti-IL1RAP antibody which binds domain 2 on IL1RAP and inhibits signaling of IL-1α, IL-1β, IL-33, IL-36α, IL-36β and IL-36γ, to varying degrees.

Blocking all six cytokines (IL-1α, IL-1β, IL-33, IL-36α, IL-36β and IL-36γ) is expected to be beneficial with regard to therapeutic approaches targeting IL-1 family of cytokine ligands and receptors. IL-1α and IL-1p are two potent pro-inflammatory cytokines and the involvement of these in acute inflammation have been extensively described. IL-33 is traditionally thought of as a Th2 cytokine that induces production of type 2 cytokines such as IL-5 and IL-13. The function of IL-36 is less well described but several studies suggest that IL-36 is involved in immune cell activation and induces release of pro-inflammatory cytokines. By targeting the pathways of all these cytokines, a more complete downregulation of the disease-related processes, for example inflammatory processes, would be achieved, which would improve therapy outcome for patients with inflammatory diseases or disorders.

To date, few treatment options address various aspects of diseases or disorders associated with interleukin-1 (IL-1) family of cytokine ligands and receptors, such as those associated with signaling of IL-1α, IL-1β, IL-33, IL-36α, IL-36β and/or IL-36γ. Consequently, there remains an urgent need for improved therapies targeting the multifaceted aspects associated with the IL-1 family of cytokine ligands and receptors.

### SUMMARY

The inventors of the present invention have generated antibodies binding to IL1RAP domain 3 with the capability of inhibiting the signaling downstream of six IL1RAP dependent ligands; IL-1α, IL-1β, IL-33, IL-36α, IL-36β and/or IL-36γ. Due to this superior property, these antibodies can be used for the prevention, treatment, alleviation, detection and/or diagnosis of diseases and disorders associated with signaling of IL-1α, IL-1β, IL-33, IL-36α, IL-36β and/or IL-36γ, thereby simultaneously targeting several downstream pathways associated with these cytokines. Consequently, these antibodies can be used in targeting several aspects and processes of diseases and disorders associated with signaling of IL-1α, IL-1β, IL-33, IL-36α, IL-36β and/or IL-36γ, for example inflammatory and fibrotic aspects in inflammatory or fibrotic diseases or disorders, or inflammatory, fibrotic or proliferative aspects in neoplastic diseases or disorders. The antibodies described here allow for simultaneous targeting of multiple cytokine pathways, for example targeting pathways of IL-1α, IL-1β, IL-33, IL-36α, IL-36β and IL-36γ, which is a clinical advantage when treating diseases or disorders where blocking only one or a few cytokines would not be effective enough to treat or prevent a disease or disorder.

A first aspect of the invention relates to an antibody or antigen-binding fragment thereof with binding specificity for interleukin-1 receptor accessory protein (IL1RAP), wherein the antibody or antigen-binding fragment comprises:
a light chain variable region comprising
   a) a CDR-L1 comprising or consisting of an amino acid sequence selected from the group consisting of ENIYSN (SEQ ID NO: 1), QSIDSY (SEQ ID NO: 7) and QSVYNDAY (SEQ ID NO: 13);
   b) a CDR-L2 comprising or consisting of an amino acid sequence selected from the group consisting of AA (SEQ ID NO: 2), GA (SEQ ID NO: 8) and LA (SEQ ID NO: 14); and
   c) a CDR-L3 comprising or consisting of an amino acid sequence selected from the group consisting of QHFWTTPRT (SEQ ID NO: 3), QCTDYSINYIGA (SEQ ID NO: 9) and QGAYYSNDWYYV (SEQ ID NO: 15);
and/or
a heavy chain variable region comprising
   d) a CDR-H1 comprising or consisting of an amino acid sequence selected from the group consisting of GFTFSDYA (SEQ ID NO: 4), GFSLSNYW (SEQ ID NO: 10) and GFSLSYD (SEQ ID NO: 16);
   e) a CDR-H2 comprising or consisting of an amino acid sequence selected from the group consisting of ITDGGSYA (SEQ ID NO: 5), ITSSDNT (SEQ ID NO: 11) and IDTSSNT (SEQ ID NO: 17); and
   f) a CDR-H3 comprising or consisting of an amino acid sequence selected from the group consisting of SRDRWPYYFDF (SEQ ID NO: 6), ARDISGINSWL (SEQ ID NO: 12) and ARGGVHAYAYAPAAFDP (SEQ ID NO: 18).

A second aspect of the invention relates to a polynucleotide encoding the antibody or antigen-binding fragment of the first aspect of the invention, or a component polypeptide chain thereof.

A third aspect of the invention relates to a vector comprising the polynucleotide according to the second aspect of the invention.

A fourth aspect of the invention relates to a recombinant host cell comprising the polynucleotide according to the second aspect of the invention or a vector according to the third aspect of the invention.

A fifth aspect of the invention relates to a method for producing the antibody or antigen-binding fragment of the first aspect of the invention, the method comprising culturing the host cell of the fourth aspect of the invention, comprising the polynucleotide of the second aspect of the invention or the vector of the third aspect of the invention, under conditions which permit expression of the encoded antibody or antigen-binding fragment thereof.

A sixth aspect of the invention relates to a pharmaceutical composition comprising
the antibody or antigen-binding fragment of the first aspect of the invention,
the polynucleotide of the second aspect of the invention,
the vector of the third aspect of the invention, and/or
the host cell of the fourth aspect of the invention,
in a pharmaceutical composition, wherein the composition further comprises a pharmaceutically-acceptable diluent, carrier or excipient.

A seventh aspect of the invention relates to
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention,
for use in medicine.

An eighth aspect of the invention relates to
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention,
for use in the prevention and/or treatment and/or alleviation and/or detection and/or diagnosis of a disease or disorder susceptible to treatment with an inhibitor of IL-1α, IL-1β, IL-33, IL-36α, IL-36β and/or IL-36γ signaling, optionally wherein the disease or disorder is associated with cells expressing IL1RAP.

A ninth aspect of the invention relates to
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention, for use in inducing cell death and/or inhibiting the growth and/or proliferation of pathological cells associated with a neoplastic disorder in a subject, or stem cells or progenitor cells thereof, wherein the cells express IL1RAP.

A tenth aspect of the invention relates to the use of
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention, in the preparation of a medicament for the prevention, treatment, alleviation, detection and/or diagnosis of a disease or disorder susceptible to treatment with an inhibitor of IL-1α, IL-1β, IL-33, IL-36α, IL-36β and/or IL-36γ signaling, optionally wherein the disease or disorder is associated with cells expressing IL1RAP.

An eleventh aspect of the invention relates to the use of
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention, in the preparation of a medicament for the detection and/or diagnosis of a disease or disorder associated with cells expressing IL1RAP.

A twelfth aspect of the invention relates to a method for the prevention and/or treatment and/or alleviation and/or detection and/or diagnosis of a disease or disorder susceptible to treatment with an inhibitor of IL-1α, IL-1β, IL-33, IL-36α, IL-36β and/or IL-36γ signaling in a subject, optionally wherein the disease or disorder is associated with cells expressing IL1RAP, comprising the step of administering to the subject an effective amount of
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention.

A thirteenth aspect of the invention relates to an in vitro method for the detection of cells expressing IL1RAP in a subject, the method comprising:
(a) providing a sample of cells from a subject to be tested, such as biopsy tissue or blood sample;
(b) optionally, extracting and/or purifying the cells present in the sample;
(c) contacting the antibody or antigen-binding fragment of the first aspect of the invention with cells present in the sample;
(d) determining whether the antibody or antigen-binding fragment thereof binds to the cells;
wherein the binding of the antibody or antigen-binding fragment thereof to the cells is indicative of the presence of a disease or disorder associated with cells expressing IL1RAP in the tissue of a subject.

A fourteenth aspect of the invention relates to an in vitro method for identifying a patient with a disease or disorder associated with cells expressing IL1RAP who would benefit from treatment with the antibody or antigen-binding fragment of the first aspect of the invention, the method comprising:
(a) providing a sample of cells, such as biopsy tissue or blood sample from a patient to be tested;
(b) optionally, extracting and/or purifying the cells present in the sample;
(c) contacting the antibody or antigen-binding fragment of the first aspect of the invention with cells present in the sample;
(d) determining whether the antibody or antigen-binding fragment thereof binds to the cells;
   wherein the binding of the antibody or antigen-binding fragment thereof to cells expressing IL1RAP is indicative of a patient who would benefit from treatment with the antibody or antigen-binding fragment of the first aspect of the invention.

A fifteenth aspect of the invention relates to a method for treating a patient with a disease or disorder associated with cells expression IL1RAP, the method comprising:
a) selecting a patient identified as having a disease or disorder associated with cells expressing IL1RAP using a method according to the fourteenth aspect of the invention; and
b) administering to said patient a therapeutic agent effective in the treatment of said disease or disorder.

A sixteenth aspect of the invention relates to a method for the detection of cells expressing IL1RAP, the method comprising:
(a) contacting an antibody or antigen-binding fragment thereof according to the first aspect with cells to be analysed for their expression of IL1RAP;
(b) determining whether the antibody or antigen-binding fragment thereof binds to the cells;
   wherein the binding of the antibody or antigen-binding fragment thereof to the cells is indicative of the presence of a disease or disorder associated with cells expressing IL1RAP in the tissue of a subject.

### DESCRIPTION OF DRAWINGS

FIG. 1: The anti-IL1RAP antibodies 43F5, 48A5 and 3G5 bind IL1RAP-expressing cells. Serial dilutions of 43F5, 48A5, 3G5 or isotype control antibody (0.2-200 nM) were added to SKMEL-5 cells (wildtype, WT). Alexa488-conjugated anti-human IgG antibody was used to detect bound antibody by flow cytometry (FIG. 1A). A similar procedure was performed with SKMEL-5 IL1RAP KO cells to which 43F5, 48A5, 3G5 or isotype control antibody were added at a single concentration, 200 nM (FIG. 1B).

FIG. 2: The anti-IL1RAP antibodies 43F5, 48A5 and 3G5 inhibit IL-1, IL-33 and IL-36 signaling. The inhibitory activity of 43F5, 48A5 and 3G5 (20 or 200 nM) on IL-1α, IL-1β, IL-33, IL-36α, IL-36β and IL-36γ signaling was evaluated in the HEK-Blue™ assay. Isotype control antibody was also included. Optical density values at 620 nm are shown for IL-1α (FIG. 2A), IL-1β (FIG. 2B), IL-33 (FIG. 2C), IL-36α (FIG. 2D), IL-36β (FIG. 2E) and IL-36γ (FIG. 2F).

FIG. 3: The anti-IL1RAP antibody 3G5 dose-dependently inhibits IL-1, IL-33 and IL-36 signaling. The inhibitory activity of 3G5 on IL-1α, IL-1β, IL-33, IL-36α, IL-36β and IL-36γ signaling was evaluated for the indicated concentrations in the HEK-Blue™ assay. Natural antagonists, i.e. IL-1Ra (IL-1α and IL-1β), ST2/IL-33R (IL-33) and IL-36Ra/IL-1F5 (IL-36α, IL-36β and IL-36γ) were included as control. Percent activity of signaling downstream of the cytokine receptor are shown for IL-1α (FIG. 3A), IL-1β (FIG. 3B), IL-33 (FIG. 3C), IL-36α (FIG. 3D), IL-36β (FIG. 3E) and IL-36γ (FIG. 3F).

FIG. 4: The anti-IL1RAP antibodies 43F5, 48A5 and 3G5 bind human and cyno IL1RAP. Cross-reactivity of 43F5, 48A5 and 3G5 to human (Homo sapiens), mouse (Mus musculus), rat (Rattus norvegicus), cynomolgus monkey (cyno; Macaca fascicularis), rabbit (Oryctolagus cuniculus), dog (Canis lupus familiaris) and pig (Sus scrofa) IL1RAP was measured by ELISA. Absorbance at 405 nm is shown.

FIG. 5: The anti-IL1RAP antibody 3G5 binds human monocytes. Human blood was stained with Alexa488-conjugated 3G5 or isotype control antibody at the indicated concentrations. Additional fluorophore-conjugated anti-CD14 and anti-CD45 antibodies were added to identify monocytes. Samples were analyzed by flow cytometry. Individual histograms are shown for the Alexa488 signal for SSC_{low}, CD45⁺ CD14⁺ cells, i.e. blood monocytes.

FIG. 6: The anti-IL1RAP antibody 3G5 inhibits IL-33 signaling in HUVEC. Human umbilical vein endothelial cells (HUVEC) were incubated with 20 μg/ml 3G5, anti-IL-33R antibody melrilimab or isotype control antibody prior to stimulation with IL-33. ELISA was performed to determine the concentration of IL-6 (FIG. 6A) and IL-8 (FIG. 6B) released in the cell supernatants in response to IL-33 stimulation.

FIG. 7: The anti-IL1RAP antibody 3G5 induces ADCC of IL1RAP-expressing cells. An ADCC assay was performed where SKMEL-5 cells were incubated with NK cells in the presence of 3G5-WT (hIgG1 wildtype), 3G5-LALA (effector-function silent hIgG1-LALA format) or isotype control antibodies at the indicated concentrations. The number of dying SKMEL-5 cells was analyzed by flow cytometry and shown as percent DAPI⁺ (dead) cells.

### DETAILED DESCRIPTION

As used herein, the singular forms “a”, “an” and “the” include plural referents unless the context clearly states otherwise. Thus, for example, reference to “an antibody” includes a plurality of such antibodies, such as one or more antibodies, at least one antibody, or two or more antibodies. Similarly, “an anti-IL1RAP antibody” can also refer to “anti-IL1RAP antibodies”, as for example the antibodies described in Examples 1-6.

The term “some embodiments” can include one, or more than one embodiment.

The use of the word “a” or “an” when used throughout the text or in conjunction with the term “comprising” in the claims and/or the specification may mean “one,” but it is also consistent with the meaning of “one or more,” “at least one,” and “one or more than one.”

### IL1RAP Antibody

A first aspect of the invention relates to an antibody or antigen-binding fragment thereof with binding specificity for interleukin-1 receptor accessory protein (IL1RAP), wherein the antibody or antigen-binding fragment comprises:
a light chain variable region comprising
   a) a CDR-L1 comprising or consisting of an amino acid sequence selected from the group consisting of ENIYSN (SEQ ID NO: 1), QSIDSY (SEQ ID NO: 7) and QSVYNDAY (SEQ ID NO: 13);
   b) a CDR-L2 comprising or consisting of an amino acid sequence selected from the group consisting of AA (SEQ ID NO: 2), GA (SEQ ID NO: 8) and LA (SEQ ID NO: 14); and
   c) a CDR-L3 comprising or consisting of an amino acid sequence selected from the group consisting of QHFWTTPRT (SEQ ID NO: 3), QCTDYSINYIGA (SEQ ID NO: 9) and QGAYYSNDWYYV (SEQ ID NO: 15);
and/or
a heavy chain variable region comprising
   d) a CDR-H1 comprising or consisting of an amino acid sequence selected from the group consisting of GFTFSDYA (SEQ ID NO: 4), GFSLSNYW (SEQ ID NO: 10) and GFSLSYD (SEQ ID NO: 16);
   e) a CDR-H2 comprising or consisting of an amino acid sequence selected from the group consisting of ITDGGSYA (SEQ ID NO: 5), ITSSDNT (SEQ ID NO: 11) and IDTSSNT (SEQ ID NO: 17); and
   f) a CDR-H3 comprising or consisting of an amino acid sequence selected from the group consisting of SRDRWPYYFDF (SEQ ID NO: 6), ARDISGINSWL (SEQ ID NO: 12) and ARGGVHAYAYAPAAFDP (SEQ ID NO: 18).

In some embodiments, the antibody or antigen-binding fragment thereof with binding specificity for IL1RAP comprises:
a light chain variable region comprising
a) a CDR-L1 comprising or consisting of an amino acid sequence selected from the group consisting of ENIYSN (SEQ ID NO: 1), QSIDSY (SEQ ID NO: 7) and QSVYNDAY (SEQ ID NO: 13);
b) a CDR-L2 comprising or consisting of an amino acid sequence selected from the group consisting of AA (SEQ ID NO: 2), GA (SEQ ID NO: 8) and LA (SEQ ID NO: 14); and
c) a CDR-L3 comprising or consisting of an amino acid sequence selected from the group consisting of QHFWTTPRT (SEQ ID NO: 3), QCTDYSINYIGA (SEQ ID NO: 9) and QGAYYSNDWYYV (SEQ ID NO: 15);
   and/or
   a heavy chain variable region comprising
d) a CDR-H1 comprising or consisting of an amino acid sequence selected from the group consisting of GFTFSDYA (SEQ ID NO: 4), GFSLSNYW (SEQ ID NO: 10) and GFSLSYD (SEQ ID NO: 16);
e) a CDR-H2 comprising or consisting of an amino acid sequence selected from the group consisting of ITDGGSYA (SEQ ID NO: 5), ITSSDNT (SEQ ID NO: 11) and IDTSSNT (SEQ ID NO: 17); and
f) a CDR-H3 comprising or consisting of an amino acid sequence selected from the group consisting of SRDRWPYYFDF (SEQ ID NO: 6), ARDISGINSWL (SEQ ID NO: 12) and ARGGVHAYAYAPAAFDP (SEQ ID NO: 18).

The terms “interleukin-1 receptor accessory protein”, “IL1RAP” and “IL1-RAP” as used herein specifically include the human IL1RAP protein, for example as described in GenBank Accession No. AAB84059, NCBI Reference Sequence: NP_002173.1 and UniProtKB/Swiss-Prot Accession No. Q9NPH3-1. IL1 RAP is also known in the scientific literature as IL R3, C3orf13, FLJ37788, IL-1RAcP and EG3556.

The terms “3G5”, “43F5” and “48A5” as used herein refer to an antibody against human IL1RAP, notwithstanding that the antibody may also bind to IL1 RAP from other species. For example, it will be understood from Example 2 that these antibodies are cross-reactive to IL1RAP from multiple species, including human and cynomolgus monkey.

It will be appreciated that “the antibody or antigen-binding fragment of the invention” can be referred to as “the polypeptide of the invention” or “the antibody polypeptide, or antigen-binding fragment thereof”, since an antibody and fragments thereof are polypeptides.

The antibody or antigen-binding fragment of the invention has specificity for IL1RAP. By “specificity” it is meant that the antibody or antigen-binding fragment is capable of binding to IL1RAP in vivo, i.e. under the physiological conditions in which IL1RAP exists within the human body. Preferably, the antibody or antigen-binding fragment does not bind to any other protein in vivo. Alternatively, it is meant that the antibody or antigen-binding fragment is capable of binding to IL1RAP ex vivo or in vitro. Such binding specificity may be determined by methods well known in the art, such as ELISA, immunohistochemistry, immunoprecipitation, Western blots and flow cytometry using transfected cells expressing IL1RAP. Advantageously, the antibody or antigen-binding fragment is capable of binding selectively to IL1RAP, i.e. it binds at least 10-fold more strongly to IL1RAP than to any other proteins.

In some embodiments, the antibody or antigen-binding fragment thereof with binding specificity for IL1RAP binds to human IL1RAP. In some embodiments, the antibody or antigen-binding fragment thereof with binding specificity for IL1RAP binds to non-human IL1RAP. In some embodiments, the antibody or antigen-binding fragment thereof with binding specificity for IL1RAP binds to IL1RAP from mouse. In some embodiments, the antibody or antigen-binding fragment thereof with binding specificity for IL1RAP binds to IL1RAP from rat. In some embodiments, the antibody or antigen-binding fragment thereof with binding specificity for IL1RAP binds to IL1RAP from cynomolgus monkey. In some embodiments, the antibody or antigen-binding fragment thereof with binding specificity for IL1RAP binds to IL1RAP from dog. In some embodiments, the antibody or antigen-binding fragment thereof with binding specificity for IL1RAP binds to IL1RAP from pig.

In some embodiments, the antibody (for example, 43F5) is cross-reactive to human, mouse, rat, cynomolgus monkey, dog and pig. In some embodiments, the antibody (for example, 48A5) is cross-reactive to human, cynomolgus monkey and dog. In some embodiments, the antibody (for example, 3G5) is cross-reactive to human and cynomolgus monkey.

In some embodiments, the antibody or antigen-binding fragment thereof with binding specificity for IL1RAP binds IL1RAP expressed on the surface of a cell. In some embodiments, the antibody or antigen-binding fragment thereof with binding specificity for IL1RAP binds to an epitope on the extracellular domain of IL1RAP. In some embodiments, the antibody or antigen-binding fragment thereof with binding specificity for IL1RAP binds to soluble IL1RAP. In some embodiments, the antibody or antigen-binding fragment thereof with binding specificity for IL1RAP binds to domain 3 of IL1RAP.

In some embodiments, the antibody or antigen-binding fragment thereof with binding specificity for IL1RAP binds IL1RAP at or within the amino acids 235 to 367 of IL1RAP.

Thus, the antibody or antigen-binding fragment may be capable of binding to an epitope located at/within domain 3 of IL1RAP (see Wang et al., 2010, Nature Immunology, 11:905-912, the disclosures of which are incorporated herein by reference), i.e. within amino acids 242 to 348 of IL1RAP (see Accession No. Q9NPH3 within UniProtKB/Swiss-Prot). For example, the epitope to which the antibody or antigen-binding fragment may be located within amino acids 239 to 251, 263 to 277, 292 to 306 or 323 to 343 of IL1RAP. However, it will be appreciated that the epitope may be non-linear.

In a further embodiment, as discussed above, the antibody or antigen-binding fragment of the invention comprises or consists of an antibody mimic selected from the group comprising or consisting of affibodies, tetranectins (CTLDs), adnectins (monobodies), anticalins, DARPins (ankyrins), avimers, iMabs, microbodies, peptide aptamers, Kunitz domains and affilins.

The term “an antibody or an antigen-binding fragment thereof” includes substantially intact antibodies as well as fragments and derivatives of antibodies. An intact antibody can be regarded as an antibody comprising variable light regions, variable heavy regions, constant light regions and constant heavy regions. The term further includes chimeric antibodies, humanised antibodies, isolated human antibodies, single chain antibodies, bispecific antibodies, multispecific antibodies, antibody heavy chains, antibody light chains, homodimers and heterodimers of antibody heavy and/or light chains, and antigen-binding fragments and derivatives of the same. Suitable antigen-binding fragments and derivatives include, but are not necessarily limited to, Fv fragments (e.g. single chain Fv and disulphide-bonded Fv), Fab-like fragments (e.g. Fab fragments, Fab′ fragments and F(ab)₂ fragments), single variable domains (e.g. V_{H} and V_{L} domains) and domain antibodies (dAbs, including single and dual formats [i.e. dAb-linker-dAb]).

The potential advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the facile production of large amounts of the said fragments.

The phrase “an antibody or an antigen-binding fragment thereof” is also intended to encompass antibody mimics (for example, non-antibody scaffold structures that have a high degree of stability yet allow variability to be introduced at certain positions). Those skilled in the art of biochemistry will be familiar with many such molecules, as discussed in Gebauer & Skerra, 2009, Curr Opin Chem Biol 13(3): 245-255 (the disclosures of which are incorporated herein by reference). Exemplary antibody mimics include: affibodies (also called Trinectins; Nygren, 2008, FEBS J, 275, 2668-2676); CTLDs (also called Tetranectins; Innovations Pharmac. Technol. (2006), 27-30); adnectins (also called monobodies; Meth. Mol. Biol., 352 (2007), 95-109); anticalins (Drug Discovery Today (2005), 10, 23-33); DARPins (ankyrins; Nat. Biotechnol. (2004), 22, 575-582); avimers (Nat. Biotechnol. (2005), 23, 1556-1561); microbodies (FEBS J, (2007), 274, 86-95); peptide aptamers (Expert. Opin. Biol. Ther. (2005), 5, 783-797); Kunitz domains (J. Pharmacol. Exp. Ther. (2006) 318, 803-809); affilins (Trends. Biotechnol. (2005), 23, 514-522); affimers (Avacta Life Sciences, Wetherby, UK).

Also included within the scope of the invention are chimeric T cell receptors (also known as chimeric T cell receptors, chimeric immunoreceptors, and chimeric antigen receptors or CARs) (see Pule et al., 2003, Cytotherapy 5(3):211-26, the disclosures of which are incorporated herein by reference). These are engineered receptors, which graft an arbitrary specificity onto an immune effector cell. Typically, CARs are used to graft the specificity of a monoclonal antibody onto a T cell; with transfer of their coding sequence facilitated by retroviral vectors. The most common form of such molecules is fusions comprising a single-chain variable fragment (scFv) derived from a monoclonal antibody fused to CD3-zeta transmembrane and endodomain. When T cells express this fusion molecule, they recognize and kill target cells that express the transferred monoclonal antibody specificity.

Persons skilled in the art will further appreciate that the invention also encompasses modified versions of antibodies and antigen-binding fragments thereof, whether existing now or in the future, e.g. modified by the covalent attachment of polyethylene glycol or another suitable polymer (see below).

Methods of generating antibodies and antibody fragments are well known in the art. For example, antibodies may be generated via any one of several methods which employ induction of in vivo production of antibody molecules, screening of immunoglobulin libraries (Orlandi. et al, 1989. Proc. Natl. Acad. Sci. U.S.A. 86:3833-3837; Winter et al., 1991, Nature 349:293-299, the disclosures of which are incorporated herein by reference) or generation of monoclonal antibody molecules by cell lines in culture. These include, but are not limited to, the hybridoma technique, the human B-cell hybridoma technique, and the Epstein-Barr virus (EBV)-hybridoma technique (Kohler et al., 1975. Nature 256:4950497; Kozbor et al., 1985. J. Immunol. Methods 81:31-42; Cote et al., 1983. Proc. Natl. Acad. Sci. USA 80:2026-2030; Cole et al., 1984. Mol. Cell. Biol. 62:109-120, the disclosures of which are incorporated herein by reference).

Suitable methods for the production of monoclonal antibodies are also disclosed in “Monoclonal Antibodies: A manual of techniques”, H Zola (CRC Press, 1988, the disclosures of which are incorporated herein by reference) and in “Monoclonal Hybridoma Antibodies: Techniques and Applications”, J G R Hurrell (CRC Press, 1982, the disclosures of which are incorporated herein by reference).

Likewise, antibody fragments can be obtained using methods well known in the art (see, for example, Harlow & Lane, 1988, “Antibodies: A Laboratory Manual”, Cold Spring Harbor Laboratory, New York, the disclosures of which are incorporated herein by reference). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

The term “amino acid” as used herein includes the standard twenty genetically-encoded amino acids and their corresponding stereoisomers in the D′ form (as compared to the natural ‘L’ form), omega-amino acids and other naturally-occurring amino acids, unconventional amino acids (e.g. α,α-disubstituted amino acids, N-alkyl amino acids, etc.) and chemically derivatised amino acids (see below).

When an amino acid is being specifically enumerated, such as “alanine” or “Ala” or “A”, the term refers to both L-alanine and D-alanine unless explicitly stated otherwise. Other unconventional amino acids may also be suitable components for polypeptides (the antibody or antigen-binding fragment thereof) of the present invention, as long as the desired functional property is retained by the antibody or antigen-binding fragment. For the amino acid sequences shown, each encoded amino acid residue, where appropriate, is represented by a single letter designation, corresponding to the trivial name of the conventional amino acid.

In some embodiments, the antibody or antigen-binding fragment thereof of the present invention and as defined herein comprises or consists of L-amino acids.

It will be appreciated by persons skilled in the art that for human therapy, human or humanised antibodies are preferably used. Humanised forms of non-human antibodies (e.g. rabbit antibodies) are genetically engineered chimeric antibodies or antibody fragments having preferably minimal-portions derived from non-human antibodies. Humanised antibodies include antibodies in which complementary determining regions of a human antibody (recipient antibody) are replaced by residues from a complementary determining region of a non-human species (donor antibody) such as mouse, rat or rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence.

Humanised antibodies optimally also include at least a portion of an antibody constant region, such as an Fc region, typically derived from a human antibody. As discussed elsewhere herein, humanised antibodies may lack cross-reactivity to mouse IL1RAP. As such, suitable surrogates that exhibit similar functional properties to the humanized antibodies can be used in murine disease models, where the surrogate results could be interpreted to reflect the functionality of the humanised antibody.

Methods for humanising non-human antibodies are well known in the art, with it being routine procedure to humanise while retaining the characteristics of the parent antibody. Generally, the humanised antibody has one or more amino acid residues introduced into it from a source which is non-human. These non-human amino acid residues, often referred to as imported residues, are typically taken from an imported variable domain. Humanisation can be essentially performed by substituting human complementarity determining regions with corresponding rodent complementarity determining regions. Accordingly, such humanised antibodies are chimeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies.

Human antibodies can also be identified using various techniques known in the art, including phage display libraries.

The optimization of antibodies, for example by humanization (see, for example, Jones et al., 1986, Nature 321:522-525; Reichmann et al., 1988. Nature 332:323-327; Verhoeyen et al., 1988, Science 239:1534-15361; U.S. Pat. No. 4,816,567, the disclosures of which are incorporated herein by reference) and/or de-immunization, as for example described in Jones et al. (Methods Mol Biol. 2009; 525:405-23), leads to the generation of antibody variants with fine-tuned properties.

### CDRs

The antibodies of the present invention are defined by their characteristic complementarity-determining region (CDR) sequences. There are several approaches for defining the CDR sequences of an antibody. The CDRs of the antibodies of the present invention have been defined according to IMGT, a numbering system well-known in the art.

The person skilled in the art will appreciate that a set of 6 CDRs (CDR-L1, CDR-L2, CDR-L3, CDR-H1, CDR-H2, CDR-H3) may be defined according to IMGT or other approaches known in the art, for example by Kabat.

Further, the person skilled in the art will appreciate that it is possible to define the CDRs of the antibody of the invention by further approaches known in the art, for example by definition of CDRs according to Chothia (AI-Lazikani et al., (1997) JMB 273,927-948), Martin (Enhanced Chothia), Gelfand or Honneger. Further approaches exist and are known in the art, such as the AbM definition (combination of Kabat definition and Chothia definition used by Oxford Molecular's AbM antibody modelling software) or the contact definition (based on analysis of crystal structures). See, e.g., Kabat et al. (Sequences of Proteins of Immunological Interest, 1987 and 1991, NIH, Bethesda, Md.), Lefranc et al. (IMGT unique numbering for immunoglobulin and T cell receptor constant domains and Ig superfamily C-like domains, Dev Comp Immunol. 2005; 29(3):185-203) and Dondelinger et al. (Understanding the Significance and Implications of Antibody Numbering and Antigen-Binding Surface/Residue Definition, Front. Immunol., 16 Oct. 2018).

The person skilled in the art could, when provided with the IMGT CDRs as presented herein, use known information to list other CDR naming conventions or approaches (e.g., Kabat or Chothia). Thus, all CDR naming conventions or approaches are encompassed.

In some embodiments, the antibody or antigen-binding fragment thereof with binding specificity for interleukin-1 receptor accessory protein (IL1RAP) comprises:
a light chain variable region comprising
   a) a CDR-L1 comprising or consisting of an amino acid sequence selected from the group consisting of ENIYSN (SEQ ID NO: 1), QSIDSY (SEQ ID NO: 7) and QSVYNDAY (SEQ ID NO: 13);
   b) a CDR-L2 comprising or consisting of an amino acid sequence selected from the group consisting of AA (SEQ ID NO: 2), GA (SEQ ID NO: 8) and LA (SEQ ID NO: 14); and
   c) a CDR-L3 comprising or consisting of an amino acid sequence selected from the group consisting of QHFWTTPRT (SEQ ID NO: 3), QCTDYSINYIGA (SEQ ID NO: 9) and QGAYYSNDWYYV (SEQ ID NO: 15);
and/or
a heavy chain variable region comprising
   d) a CDR-H1 comprising or consisting of an amino acid sequence selected from the group consisting of GFTFSDYA (SEQ ID NO: 4), GFSLSNYW (SEQ ID NO: 10) and GFSLSYD (SEQ ID NO: 16);
   e) a CDR-H2 comprising or consisting of an amino acid sequence selected from the group consisting of ITDGGSYA (SEQ ID NO: 5), ITSSDNT (SEQ ID NO: 11) and IDTSSNT (SEQ ID NO: 17); and
   f) a CDR-H3 comprising or consisting of an amino acid sequence selected from the group consisting of SRDRWPYYFDF (SEQ ID NO: 6), ARDISGINSWL (SEQ ID NO: 12) and ARGGVHAYAYAPAAFDP (SEQ ID NO: 18).

However, the person skilled in the art will appreciate that a low level of mutation (typically, just one or two amino acids) within a CDR sequence may be tolerated without loss of the specificity of the antibody or antigen-binding fragment for IL1RAP.

In some embodiments, the antibody or antigen-binding fragment thereof with binding specificity for IL1RAP comprises CDRs as described above (comprising or consisting of the amino acid sequence selected from the group consisting of SEQ ID NOs 1 to 18), wherein any one of the amino acids of the CDRs has been altered for another amino acid, for example, with the proviso that no more than 2 amino acids have been so altered, such as 1 amino acid.

### Light Chain Variable Regions

As elaborated in Example 1, rabbits or mice were immunized with human and murine IL1RAP. Subsequently, resulting antibodies were analysed for desired properties, such as binding to IL1RAP and capacity to inhibit signaling of IL-1α, IL-1β, IL-33, IL-36α, IL-36β and/or IL-36γ. 3G5, 43F5 and 48A5 were identified as antibodies with superior properties with regard to these features, and may subsequently be modified and optimized with the aim to improve the antibodies for clinical and therapeutic application. This modification or optimization procedure may result in antibody variants harbouring variable light chain regions and variable heavy chain regions with different amino acid sequences. However, the CDRs would be identical for all modified or optimized antibody variants.

In some embodiments, the antibody or antigen-binding fragment thereof with binding specificity for IL1RAP comprises a light chain variable region comprising the CDRs comprising or consisting of an amino acid sequence selected from the group consisting of ENIYSN (SEQ ID NO: 1), QSIDSY (SEQ ID NO: 7) and QSVYNDAY (SEQ ID NO: 13); comprising or consisting of an amino acid sequence selected from the group consisting of AA (SEQ ID NO: 2), GA (SEQ ID NO: 8) and LA (SEQ ID NO: 14); and/or comprising or consisting of an amino acid sequence selected from the group consisting of QHFWTTPRT (SEQ ID NO: 3), QCTDYSINYIGA (SEQ ID NO: 9) and QGAYYSNDWYYV (SEQ ID NO: 15).

In some embodiments, the antibody or antigen-binding fragment thereof with binding specificity for IL1RAP comprises a light chain variable region comprising the CDRs comprising
a) a CDR-L1 comprising or consisting of the amino acid sequence of ENIYSN (SEQ ID NO: 1); a CDR-L2 comprising or consisting of the amino acid sequence of AA (SEQ ID NO: 2); and a CDR-L3 comprising or consisting of the amino acid sequence of QHFWTTPRT (SEQ ID NO: 3);
b) a CDR-L1 comprising or consisting of the amino acid sequence of QSIDSY (SEQ ID NO: 7); a CDR-L2 comprising or consisting of the amino acid sequence of GA (SEQ ID NO: 8); and a CDR-L3 comprising or consisting of the amino acid sequence of QCTDYSINYIGA (SEQ ID NO: 9); or
c) a CDR-L1 comprising or consisting of the amino acid sequence of QSVYNDAY (SEQ ID NO: 13); a CDR-L2 comprising or consisting of the amino acid sequence of LA (SEQ ID NO: 14); and a CDR-L3 comprising or consisting of the amino acid sequence of QGAYYSNDWYYV (SEQ ID NO: 15).

In some embodiments, the antibody or antigen-binding fragment thereof with binding specificity for IL1RAP comprises a light chain variable region comprising or consisting of an amino acid sequence selected from the group consisting of SEQ ID NO: 19, SEQ ID NO: 21, and SEQ ID NO: 23; or an amino acid sequence having at least 70% sequence identity thereto, for example at least 80%, 85%, 90%, 95%, 98% or 99% sequence identity. These light chain variable regions may be, for example, part of humanized 3G5, 43F5 and 48A5 antibody variants, respectively.

Percent identity (or sequence identity) can be determined by, for example, the LALIGN program at the Expasy facility site (http://www.ch.embnet.org/software/LALIGN_form.html) using as parameters the global alignment option, scoring matrix BLOSUM62, opening gap penalty −14, extending gap penalty −4. Alternatively, the percent sequence identity between two polypeptides, such as parts of an antibody, may be determined using suitable computer programs, for example the GAP program of the University of Wisconsin Genetic Computing Group and it will be appreciated that percent identity is calculated in relation to polypeptides whose sequence has been aligned optimally.

The alignment may alternatively be carried out using the ClustalW program. The parameters used may be as follows:
Fast pair-wise alignment parameters: K-tuple(word) size; 1, window size; 5, gap penalty; 3, number of top diagonals; 5. Scoring method: x percent.
Multiple alignment parameters: gap open penalty; 10, gap extension penalty; 0.05.
Scoring matrix: BLOSUM.

Alternatively, the BESTFIT program may be used to determine local sequence alignments.

The person skilled in the art will consider further alterations to the above described light chain variable regions, for example to further optimize the antibody or antigen-binding fragment. For example, the person skilled in the art will, as done during humanization and de-immunization procedures, consider altering amino acids in the framework regions, i.e. outside the epitope binding CDR regions, thereby not altering the CDR regions.

In some embodiments, the antibody or antigen-binding fragment thereof with binding specificity for IL1RAP comprises a light chain variable region as described above, wherein any one of the amino acids of the framework region of the light chain variable region has been altered for another amino acid, with the proviso that no more than 5 amino acids have been so altered, such as 4 amino acids, no more than 3 amino acids, such as 2 amino acids or no more than 1 amino acid.

### Heavy Chain Variable Regions

In some embodiments, the antibody or antigen-binding fragment thereof with binding specificity for IL1RAP comprises a heavy chain variable region comprising the CDRs comprising or consisting of an amino acid sequence selected from the group consisting of GFTFSDYA (SEQ ID NO: 4), GFSLSNYW (SEQ ID NO: 10) and GFSLSYD (SEQ ID NO: 16); comprising or consisting of an amino acid sequence selected from the group consisting of ITDGGSYA (SEQ ID NO: 5), ITSSDNT (SEQ ID NO: 11) and IDTSSNT (SEQ ID NO: 17); and comprising or consisting of an amino acid sequence selected from the group consisting of SRDRWPYYFDF (SEQ ID NO: 6), ARDISGINSWL (SEQ ID NO: 12) and ARGGVHAYAYAPAAFDP (SEQ ID NO: 18).

In some embodiments, the antibody or antigen-binding fragment thereof with binding specificity for IL1RAP comprises a heavy chain variable region comprising
a) a CDR-H1 comprising or consisting of the amino acid sequence of GFTFSDYA (SEQ ID NO: 4); a CDR-H2 comprising or consisting of the amino acid sequence of ITDGGSYA (SEQ ID NO: 5); and a CDR-H3 comprising or consisting of the amino acid sequence of SRDRWPYYFDF (SEQ ID NO: 6);
b) a CDR-H1 comprising or consisting of the amino acid sequence of GFSLSNYW (SEQ ID NO: 10); a CDR-H2 comprising or consisting of the amino acid sequence of ITSSDNT (SEQ ID NO: 11); and a CDR-H3 comprising or consisting of the amino acid sequence of ARDISGINSVVL (SEQ ID NO: 12); or
c) a CDR-H1 comprising or consisting of the amino acid sequence of GFSLSYD (SEQ ID NO: 16); a CDR-H2 comprising or consisting of the amino acid sequence of IDTSSNT (SEQ ID NO: 17); and a CDR-H3 comprising or consisting of the amino acid sequence of ARGGVHAYAYAPAAFDP (SEQ ID NO: 18).

In some embodiments, the antibody or antigen-binding fragment thereof with binding specificity for IL1RAP comprises a heavy chain variable region comprising or consisting of an amino acid sequence selected from the group consisting of SEQ ID NO: 20, SEQ ID NO: 22, and SEQ ID NO: 24; or an amino acid sequence having at least 70% sequence identity thereto, for example at least 80%, 85%, 90%, 95%, 98% or 99% sequence identity. These heavy chain variable regions may be, for example, part of humanized 3G5, 43F5 and 48A5 antibody variants, respectively.

The person skilled in the art will consider further alterations to the above described heavy chain variable regions, for example to further optimize the antibody or antigen-binding fragment. For example, the person skilled in the art will, as done during humanization and de-immunization procedures, consider altering amino acids in the framework regions, i.e. outside the epitope binding CDR regions, thereby not altering the CDR regions.

In some embodiments, the antibody or antigen-binding fragment thereof with binding specificity for IL1RAP comprises a heavy chain variable region as described above, wherein any one of the amino acids of the framework region of the heavy chain variable region has been altered for another amino acid, with the proviso that no more than 5 amino acids have been so altered, such as 4 amino acids, no more than 3 amino acids, such as 2 amino acids or no more than 1 amino acid.

### Combination of Variable Light and Variable Heavy Chains

The person skilled in the art will appreciate that any of the above described variants of light chain variable regions can be combined with any of the above described variants of heavy chain variable regions.

In some embodiments, the antibody or antigen-binding fragment thereof with binding specificity for IL1RAP comprises a light chain variable region which comprises or consists of the amino acid sequence of SEQ ID NO: 19 and a heavy chain variable region which comprises or consists of the amino acid sequence of SEQ ID NO: 20, or an amino acid sequence having at least 70% sequence identity to SEQ ID NO: 19 or SEQ ID NO: 20, for example at least 80%, 85%, 90%, 95%, 98% or 99% sequence identity.

In some embodiments, the antibody or antigen-binding fragment thereof with binding specificity for IL1RAP comprises:
a) a light chain variable region which comprises or consists of the amino acid sequence of SEQ ID NO: 19 and a heavy chain variable region which comprises or consists of the amino acid sequence of SEQ ID NO: 20;
b) a light chain variable region which comprises or consists of the amino acid sequence of SEQ ID NO: 19 and a heavy chain variable region which comprises or consists of the amino acid sequence of SEQ ID NO: 22;
c) a light chain variable region which comprises or consists of the amino acid sequence of SEQ ID NO: 19 and a heavy chain variable region which comprises or consists of the amino acid sequence of SEQ ID NO: 24;
d) a light chain variable region which comprises or consists of the amino acid sequence of SEQ ID NO: 21 and a heavy chain variable region which comprises or consists of the amino acid sequence of SEQ ID NO: 20;
e) a light chain variable region which comprises or consists of the amino acid sequence of SEQ ID NO: 21 and a heavy chain variable region which comprises or consists of the amino acid sequence of SEQ ID NO: 22;
f) a light chain variable region which comprises or consists of the amino acid sequence of SEQ ID NO: 21 and a heavy chain variable region which comprises or consists of the amino acid sequence of SEQ ID NO: 24;
g) a light chain variable region which comprises or consists of the amino acid sequence of SEQ ID NO: 23 and a heavy chain variable region which comprises or consists of the amino acid sequence of SEQ ID NO: 20;
h) a light chain variable region which comprises or consists of the amino acid sequence of SEQ ID NO: 23 and a heavy chain variable region which comprises or consists of the amino acid sequence of SEQ ID NO: 22; or
i) a light chain variable region which comprises or consists of the amino acid sequence of SEQ ID NO: 23 and a heavy chain variable region which comprises or consists of the amino acid sequence of SEQ ID NO: 24;
   or an amino acid sequence having at least 70% sequence identity thereto, for example at least 80%, 85%, 90%, 95%, 98% or 99% sequence identity.

It will be appreciated by persons skilled in the art that the above-defined light chain variable regions, heavy chain variable regions and combinations thereof may further be combined with a light/heavy chain constant regions, or parts thereof (see below).

In some embodiments, the antibody or antigen-binding fragment thereof with binding specificity for IL1RAP comprises a heavy chain variable region as described above, wherein any one of the amino acids of the framework region of the light chain variable region and/or the heavy chain variable region has been altered for another amino acid, with the proviso that no more than 5 amino acids have been so altered, such as 4 amino acids, no more than 3 amino acids, such as 2 amino acids or no more than 1 amino acid.

### Constant Regions and Fc Part

The person skilled in the art will appreciate that any light constant region known in the art can be combined with any of the above described variants of light variable regions, and that any heavy constant region known in the art can be combined with any of the above described variants of heavy variable regions, to form a full antibody.

In some embodiments, the antibody or antigen-binding fragment thereof with binding specificity for IL1RAP comprises a light chain constant region, or part thereof. In some embodiments, the antibody or antigen-binding fragment thereof with binding specificity for IL1RAP comprises a light chain constant region which is of a kappa or lambda light chain. In some embodiments, the antibody or antigen-binding fragment thereof with binding specificity for IL1RAP comprises a kappa light chain. In some embodiments, the antibody or antigen-binding fragment thereof with binding specificity for IL1RAP comprises a lambda light chain.

In some embodiments, the antibody or antigen-binding fragment thereof with binding specificity for IL1RAP comprises a light chain constant region comprising or consisting of the amino acid sequence of SEQ ID NO: 35, or an amino acid sequence having at least 70% sequence identity to SEQ ID NO: 35, for example at least 80%, 85%, 90%, 95%, 98% or 99% sequence identity.

In some embodiments, the antibody or antigen-binding fragment thereof with binding specificity for IL1RAP comprises a heavy chain constant region, or part thereof. In some embodiments, the antibody or antigen-binding fragment thereof with binding specificity for IL1RAP comprises a heavy chain constant region which is selected from the group consisting of α, δ, γ, ε and μ. In some embodiments, the antibody or antigen-binding fragment thereof with binding specificity for IL1RAP comprises a heavy chain constant region which is of an immunoglobulin isotype selected from the group consisting of IgA, IgD, IgG, IgE and IgM.

In some embodiments, the antibody or antigen-binding fragment thereof with binding specificity for IL1RAP comprises a heavy chain constant region which is of an IgG immunoglobulin isotype. In some embodiments, the antibody or antigen-binding fragment thereof with binding specificity for IL1RAP is an IgG antibody. In some embodiments, the antibody or antigen-binding fragment thereof with binding specificity for IL1RAP comprises a heavy chain constant region which is of an immunoglobulin subclass selected from the group consisting of IgG1, IgG2, IgG3 and IgG4. In some embodiments, the antibody or antigen-binding fragment thereof with binding specificity for IL1RAP is an IgG1 antibody. In some embodiments, the antibody or antigen-binding fragment thereof with binding specificity for IL1RAP is an IgG2 antibody. In some embodiments, the antibody or antigen-binding fragment thereof with binding specificity for IL1RAP is an IgG3 antibody. In some embodiments, the antibody or antigen-binding fragment thereof with binding specificity for IL1RAP is an IgG4 antibody.

In some embodiments, the antibody or antigen-binding fragment thereof with binding specificity for IL1RAP comprises or consists of a heavy chain constant region comprising or consisting of the amino acid sequence of SEQ ID NO: 36 or SEQ ID NO: 37, or an amino acid sequence having at least 70% sequence identity to SEQ ID NO: 36 or SEQ ID NO: 37, for example at least 80%, 85%, 90%, 95%, 98% or 99% sequence identity.

In some embodiments the antibody or antigen-binding fragment thereof with binding specificity for IL1RAP comprises or consists of a light chain constant region and/or a heavy chain constant region wherein any one of the amino acids of the above mentioned light chain constant region and/or above mentioned heavy chain constant region has been altered for another amino acid, with the proviso that no more than 5 amino acids have been so altered, such as 4 amino acids, no more than 3 amino acids, such as 2 amino acids or no more than 1 amino acid.

In some embodiments, the antibody or antigen-binding fragment of the present invention comprises a CH1, CH2 and/or CH3 region of an IgG heavy chain (such as an IgG1, IgG2, IgG3 or IgG4 heavy chain). Thus, the antibody or antigen-binding fragment may comprise part or all of the constant regions from an IgG1 heavy chain. For example, the antibody or antigen-binding fragment may be a Fab fragment comprising CH1 and CL constant regions, combined with any of the above-defined heavy and light variable regions, respectively.

Likewise, the above-defined antibodies or antigen-binding fragments of the invention may further comprise a light chain constant region, or part thereof. For example, the antibody or antigen-binding fragment may comprise a CL region from a kappa or lambda light chain.

In some embodiments, the antibody or antigen-binding fragment thereof with binding specificity for IL1RAP comprises or consists of
any one of the above mentioned light chain variable regions; and/or
any one of the above mentioned heavy chain variable regions; and/or
any one of the above mentioned light chain constant regions; and/or
any one of the above mentioned heavy chain constant regions.

In some embodiments, the antibody or antigen-binding fragment thereof with binding specificity for IL1RAP comprises
a light chain variable region comprising or consisting of the amino acid sequence selected from the group consisting of an amino acid sequence of SEQ ID NOs: 19, 21, and 23; and/or
a heavy chain variable region comprising or consisting of an amino acid sequence selected from the group consisting of SEQ ID NOs: 20, 22, and 24; and/or
a light chain constant region comprising or consisting of the amino acid sequence of SEQ ID NO: 35; and/or
a heavy chain constant region comprising or consisting of the amino acid sequence of SEQ ID NOs: 36 or 37.

In some embodiments, the antibody or antigen-binding fragment thereof with binding specificity for IL1RAP comprises an Fc region. Fc region may also be referred to as Fc domain. In some embodiments, the antibody or antigen-binding fragment thereof with binding specificity for IL1RAP comprises an Fc region which is naturally occurring. In some embodiments, the antibody or antigen-binding fragment thereof with binding specificity for IL1RAP comprises an Fc region which is non-naturally occurring. In some embodiments, the antibody or antigen-binding fragment thereof with binding specificity for IL1RAP comprises an Fc region with a modified, for example with a mutated, IgG constant region.

In some embodiments, the antibody or antigen-binding fragment thereof with binding specificity for IL1RAP comprises an Fc region wherein the Fc region comprises one or more of the mutations identified in Table 1.

The Fc region may be naturally occurring (e.g. part of an endogenously produced antibody) or may be artificial (e.g. comprising one or more point mutations relative to a naturally-occurring Fc region).

As is well documented in the art, the Fc region of an antibody mediates its serum half-life and effector functions, such as complement-dependent cytotoxicity (CDC), antibody-dependent cellular cytotoxicity (ADCC) and antibody-dependent cell phagocytosis (ADCP).

Engineering the Fc region of a therapeutic monoclonal antibody or Fc fusion protein allows the generation of molecules that are better suited to the pharmacology activity required of them (Strohl, 2009, Curr Opin Biotechnol 20(6):685-91, the disclosures of which are incorporated herein by reference).

### (a) Engineered Fc Regions for Increased Half-Life

One approach to improve the efficacy of a therapeutic antibody is to increase its serum persistence, thereby allowing higher circulating levels, less frequent administration and reduced doses.

The half-life of an IgG depends on its pH-dependent binding to the neonatal receptor FcRn. FcRn, which is expressed on the surface of endothelial cells, binds the IgG in a pH-dependent manner and protects it from degradation.

Some antibodies that selectively bind the FcRn at pH 6.0, but not pH 7.4, exhibit a higher half-life in a variety of animal models.

Several mutations located at the interface between the CH2 and CH3 domains, such as T250Q/M428L (Hinton et al., 2004, J Biol Chem. 279(8):6213-6, the disclosures of which are incorporated herein by reference) and M252Y/S254T/T256E+H433K/N434F (Vaccaro et al., 2005, Nat. Biotechnol. 23(10):1283-8, the disclosures of which are incorporated herein by reference), have been shown to increase the binding affinity to FcRn and the half-life of IgG1 in vivo.

### (b) Engineered Fc Regions for Altered Effector Function

Depending on the therapeutic antibody or Fc fusion protein application, it may be desired to either reduce or increase the effector function (such as ADCC).

For antibodies that target cell-surface molecules, especially those on immune cells, abrogating effector functions may be required for certain clinical indications.

Conversely, for antibodies intended for oncology use (such as in the treatment of leukemias and solid tumours; see below), increasing effector functions may improve the therapeutic activity.

The four human IgG isotypes bind the activating Fcγ receptors (FcγRI, FcγRIIa, FcγRIIIa), the inhibitory FcγRIIb receptor, and the first component of complement (C1q) with different affinities, yielding very different effector functions (Bruhns et al., 2009, Blood. 113(16):3716-25, the disclosures of which are incorporated herein by reference).

Binding of IgG to the FcγRs or C1q depends on residues located in the hinge region and the CH2 domain. Two regions of the CH2 domain are critical for FcγRs and C1q binding, and have unique sequences in IgG2 and IgG4. Substitutions into human IgG1 of IgG2 residues at positions 233-236 and IgG4 residues at positions 327, 330 and 331 were shown to greatly reduce ADCC and CDC (Armour et al., 1999, Eur J Immunol. 29(8):2613-24; Shields et al., 2001, J Biol Chem. 276(9):6591-604, the disclosures of which are incorporated herein by reference). The ‘LALA’ mutation, L234A/L235A, has been introduced in several therapeutic IgG1 antibodies for creation of an effector function silent Fc region, see for example Xu et al., 2000, Cell. Immuno. 200: 16-26. Furthermore, Idusogie et al. demonstrated that alanine substitution at different positions, including K322, significantly reduced complement activation (Idusogie et al., 2000, J Immunol. 164(8):4178-84, the disclosures of which are incorporated herein by reference). Similarly, mutations in the CH2 domain of murine IgG2A were shown to reduce the binding to FcγRI, and C1q (Steurer. et al., 1995. J Immunol. 155(3):1165-74, the disclosures of which are incorporated herein by reference).

Numerous mutations have been made in the CH2 domain of human IgG1 and their effect on ADCC and CDC tested in vitro (see references cited above). Notably, alanine substitution at position 333 was reported to increase both ADCC and CDC (Shields et al., 2001, supra; Steurer et al., 1995, supra). Lazar et al. described a triple mutant (S239D/I332E/A330L) with a higher affinity for FcγRIIIa and a lower affinity for FcγRIIb resulting in enhanced ADCC (Lazar et al., 2006, PNAS 103(11):4005-4010, the disclosures of which are incorporated herein by reference). The same mutations were used to generate an antibody with increased ADCC (Ryan et al., 2007, Mol. Cancer Ther. 6:3009-3018, the disclosures of which are incorporated herein by reference). Richards et al. studied a slightly different triple mutant (S239D/I332E/G236A) with improved FcγRIIIa affinity and FcγRIIa/FcγRIIb ratio that mediates enhanced phagocytosis of target cells by macrophages (Richards et al., 2008. Mol Cancer Ther. 7(8):2517-27, the disclosures of which are incorporated herein by reference).

Due to their lack of effector functions, IgG4 antibodies represent a preferred IgG subclass for receptor blocking without cell depletion (i.e. inhibition of IL-1 signaling). IgG4 molecules can exchange half-molecules in a dynamic process termed Fab-arm exchange. This phenomenon can also occur in vivo between therapeutic antibodies and endogenous IgG4.

The S228P mutation has been shown to prevent this recombination process allowing the design of less unpredictable therapeutic IgG4 antibodies (Labrijn et al., 2009, Nat Biotechnol. 27(8):767-71, the disclosures of which are incorporated herein by reference).

Examples of engineered Fc regions are shown in Table 1 below.

TABLE 1
Examples of Engineered Fc

| | | | | Effector |
|---|---|---|---|---|
| Isotype | Species | Mutations* | FcR/C1q Binding | Function |
| IgG1 | Human | T250Q/M428L¹ | Increased binding | Increased |
| | | | to FcRn | half-life |
| IgG1 | Human | M252Y/S254T/T256E + | Increased binding | Increased |
| | | H433K/N434F² | to FcRn | half-life |
| IgG1 | Human | M428L/N434S³ | Increased binding | Increased |
| | | | to FcRn | half-life |
| IgG1 | Human | E233P/L234V/L235A/?G236 + | Reduced binding | Reduced ADCC |
| | | A327G/A330S/P331S^{4, 5} | to FcγRI | and CDC |
| IgG1 | Human | S239D/S298A/I332E + | Increased binding | Increased |
| | | S239D/A330L/I332E⁶ | to FcγRIIIa | ADCC |
| IgG1 | Human | S239D/I332E⁷ | Increased binding | Increased |
| | | | to FcγRIIIa | ADCC |
| IgG1 | Human | S298A/E333A/K334A⁸ | Increased binding | Increased |
| | | | to FcγRIIIa | ADCC |
| IgG1 | Human | E333A⁹ | Increased binding | Increased |
| | | | to FcγRIIIa | ADCC |
| | | | | and CDC |
| IgG1 | Human | P257I/Q311¹⁰ | Increased binding | Unchanged |
| | | | to FcRn | half-life |
| IgG1 | Human | K326W/E333S¹¹ | Increased binding | Increased |
| | | | to C1q | CDC |
| IgG1 | Human | S239D/I332E/G236A¹² | Increased | Increased |
| | | | FcγRIIa/ | macrophage |
| | | | FcγRIIb | phagocytosis |
| | | | ratio | |
| IgG1 | Human | K322A⁸ | Reduced binding | Reduced CDC |
| | | | to C1q | |
| | | N297S | | Reduced |
| | | | | (abrogated) |
| | | | | ADCC |
| | | N297Q | | Reduced |
| | | | | (abrogated) |
| | | | | ADCC |
| | | R292P + V305I +/− | | Increased |
| | | F243L¹³ | | ADCC |
| | | P247I/A339Q¹⁴ | | Increased |
| | | | | ADCC |
| IgG4 | Human | S228P¹⁵ | — | Reduced Fab-arm |
| | | | | exchange |
| IgG2a | Mouse | L235E + E318A/K320A/K322A¹¹ | Reduced binding to | Reduced ADCC |
| | | | FcγRI and C1q | and CDC |

*The position of the Fc amino acid mutations is defined using the EU Numbering Scheme (see Edelman et al., 1969, Proc. Natl. Acad. Sci. USA, 63: 78-85), which may differ from the actual numbering in SEQ ID NOS: 36 and 37, for example, see further elaborations on the numbering of mutations below.
References to Table 1
¹Hinton et al 2004 J. Biol. Chem. 279(8): 6213-6)
²Vaccaro et al. 2005 Nat Biotechnol. 23(10): 1283-8)
³Zalevsky et al 2010 Nat. Biotechnology 28(2): 157-159
⁴Armour K. L. et al., 1999. Eur J Immunol. 29(8): 2613-24
⁵Shields R. L. et al., 2001. J Biol Chem. 276(9): 6591-604
⁶Masuda et al. 2007, Mol Immunol. 44(12): 3122-31
⁷Bushfield et al 2014, Leukemia 28(11): 2213-21
⁸Okazaki et al. 2004, J Mol Biol.; 336(5): 1239-49
⁹Idusogie et al., 2000. J Immunol. 164(8): 4178-84
¹⁰Datta-Mannan A. et al., 2007. Drug Metab. Dispos. 35: 86-94
¹¹Steurer W. et al., 1995. J Immunol. 155(3): 1165-74
¹²Richards et al. 2008 Mol Cancer There. 7(8): 2517-27
¹³U.S. Pat. No. 7,960,512 B2
¹⁴EP 2 213 683
¹⁵Labrijn A. F. et al., 2009. Nat Biotechnol. 27(8): 767-71

In some embodiments, the antibody or antigen-binding fragment thereof with binding specificity for IL1RAP comprises an Fc region wherein the Fc region comprises one or more of the mutations selected from the group consisting of L234A, L235A, P329G, G237A, P238S, H269A, A330S and P331S as defined by the EU Index. These mutations may reduce the effector function of the antibody. The EU Index is conventionally used in the art (see generally, Kabat et al, 1991).

The person skilled in the art will appreciate that the exact position of the herein described Fc mutations may differ in the antibody of the invention. This is due to species variations in chimeric antibodies (and therefrom humanized or humanized/de-immunized antibodies) due to species differences in the variable regions. Varying lengths of regions in the variable heavy region lead to a shift of amino acid positions as compared to the one in the EU Index. However, the relative position of the amino acids in the Fc part is preserved.

For example, the LALA mutation is defined by the EU index to be at position L234A, L235A.

An exemplary antibody of the present invention may
comprise a variable heavy chain region of, for
example, SEQ ID NO: 20 (or SEQ ID NO: 22 or 24)
Heavy chain variable region (non-humanized, non-
deimmunized)
EVQLVESGGVLVKPGGSLKLSCAASGFTESDYAMSWVRQTPEKRLEWVAT
ITDGGSYAYYTDDVKGRFTVSRDNARNNLFLQMSHLRSEDTGIYYCSRDR
WPYYFDFWGRGTTLTVSS
and
a constant heavy chain (not containing the
LALA mutation) of, for example, SEQ ID NO: 36
Immunoglobulin IgG1 constant heavy chain
(heavy chain constant region) (za allotype)
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKENWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
(the bold and underlined amino acid residues
indicate the residues that are altered when
the LALA-mutation is introduced as shown in
SEQ ID NO: 37, below).
Alternatively, an exemplary antibody of the
present invention may comprise a variable
heavy chain region of, for example, SEQ ID NO: 20
(or SEQ ID NO: 22 or 24)
Heavy chain variable region (non-humanized, non-
deimmunized)
EVQLVESGGVLVKPGGSLKLSCAASGFTFSDYAMSWVRQTPEKRLEWVAT
ITDGGSYAYYTDDVKGRFTVSRDNARNNLFLQMSHLRSEDTGIYYCSRDR
WPYYFDFWGRGTTLTVSS
and
a constant heavy chain (containing the LALA
mutation) of, for example, SEQ ID NO: 37
Immunoglobulin IgG1 constant heavy chain (heavy
chain constant region) (za allotype) with
‘LALA’ mutation
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKENWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG

The amino acid mutations (LL to AA in the above sequences) are at positions 237 and 238 (so L237A, L238A), and are, in the context of the amino acid sequence of the heavy chain constant region, equivalent to the L234A, L235A-mutations according to the EU index.

The person skilled in the art will appreciate that the same reasoning applies to the other named mutations that are known in the art, such as P329G, G237A, P238S, H269A, A330S and P331S as defined by the EU Index.

In some embodiments, the antibody or antigen-binding fragment thereof with binding specificity for IL1RAP comprises an Fc region, wherein the glycan attached to the Fc region is lacking fucose. In some embodiments, the antibody or antigen-binding fragment thereof with binding specificity for IL1RAP comprises an Fc region which is lacking fucose. In some embodiments, the antibody or antigen-binding fragment thereof with binding specificity for IL1RAP comprises an Fc region, wherein the glycan attached to the Fc region is low in fucose. In some embodiments, the antibody or antigen-binding fragment thereof with binding specificity for IL1RAP comprises an Fc region which is low in fucose.

In some embodiments, the antibody or antigen-binding fragment thereof with binding specificity for IL1RAP, wherein the antibody or antigen-binding fragment is produced in a FUT8 negative cell line. In some embodiments, the antibody is produced in a FUT8 negative cell line. It is possible to create FUT8 negative cell lines by knocking out the FUT8 gene, which encodes for the a-(1,6)-fucosyltransferase enzyme, which in turn catalyses fucose transfer. Antibodies comprising reduced fucosylation of the Fc region exhibit enhanced ADCC activity, as described in WO 00/61739 A1. It will be appreciated by a skilled person in the art that FUT8 negative cell lines are useful when producing antibodies with enhanced ADCC activity.

In some embodiments, the antibody or antigen-binding fragment thereof with binding specificity for IL1RAP is an intact antibody, or part of an intact antibody.

In some embodiments, the antibody or antigen-binding fragment thereof with binding specificity for IL1RAP is comprising or consisting of an antigen-binding fragment selected from the group consisting of Fv fragments (e.g. single chain Fv and disulphide-bonded Fv), Fab-like fragments (e.g. Fab fragments, Fab′ fragments and F(ab)₂ fragments) and domain antibodies (e.g. single V_{H} variable domains or V_{L} variable domains).

The section below under “Sequences” provide sequences of certain exemplary embodiments of the antibodies disclosed and claimed herein.

### Inhibition of Signaling

Interleukins are implicated in various diseases and disorders.

Interleukins, such as interleukins of the IL-1 family, as are cytokines. Cytokines (including, for example, chemokines, interferons, interleukins, lymphokines, and tumour necrosis factors) are of importance for cell signaling. In other words, cytokines have a signaling function to the cell, for example via binding to the respective receptor. For example, IL-1α, IL-1β, IL-33, IL-36α, IL-36β and/or IL-36γ signaling, as described herein, encompasses the events that these cytokines trigger on the cell surface or in the cell. On the cell surface, these signaling events affect, for example, the confirmation of the receptor, the properties of co-receptors and other molecules, the binding of molecules extracellularly, on the cell surface, in or at the cell membrane or in the cell. These events may be physiological or pathological. These events may be biological pathways, leading for example to a response in the cell as a reaction to the signaling. This response may be physiological or pathological.

As used herein “inhibition of signaling” is defined as a reduction of said signaling event, or of, for example, the activity, confirmation, or production of a receptor or biological pathway or molecule activity. This inhibition is to be understood as comparing the situation where the inhibiting factor (in the case of the present invention the antibody or antigen-binding fragment thereof with binding specificity for IL1RAP) is present compared to the situation where the inhibiting factor is not present. The person skilled in the art will appreciate that the degree of inhibition can be determined by methods well known in the art, depending on the signaling event or pathway or activity to be measured. This method can be, for example, a cell assay, for example as described in Example 2.

It is to be noted that, as used herein, phrases such as “inhibiting signaling of (for example) IL-1α” are used interchangeably with the phrase “inhibiting (for example) IL-1α signaling”.

### IL-1

Interleukin-1 (IL-1) is a potent pro-inflammatory cytokine that can be produced by a variety of cell types, including mononuclear phagocytes, in response to infection and inflammation. The IL-1 family consists of seven agonists, including IL-1α and IL-1β, and three naturally occurring receptor antagonists, including the IL-1 receptor antagonist (IL-1Ra or IL1RA) (Dinarello, C A, Blood 1996, 87(6): 2095-147). Two IL-1 receptors, IL-1R type I and IL-1R type II, have been identified. Both receptors can interact with all three forms of the IL-1 family molecules. IL-1RI is responsible for mediating IL-1-induced cellular activation. However, the IL-1/IL-1RI complex cannot signal by itself, but is dependent on association with a second receptor chain, IL-1R Accessory Protein (IL1RAP) (Dinarello, C A, Blood 1996, 87(6): 2095-147). In contrast to IL-1RI, IL-1RII does not induce cellular activation upon binding to IL-1 and thus IL-1RII functions as a regulatory decoy receptor, leading to a net decrease in IL-1 available to bind to IL-1RI.

In addition to IL1-signaling, IL1RAP is critical for mediating the effects of IL-33, through the ST2/IL1RAP complex, and IL-36, through the IL1Rrp2/IL1RAP complex (Garlanda et al, Immunity. 2013 Dec. 12; 39(6):1003-18).

IL-1α is also released from damaged cells and can act as an alarmin. IL-1 is a potent pro-inflammatory cytokine, which is induced at sites of local infection or inflammation and is involved in the regulation of a variety of physiological and cellular events (summarised in Dinarello C A, CHEST, 2000, 118: 503-508 and Dinarello, C A, Clin Exp Rheumatol, 2002, 20(5 Suppl 27): S1-13). It is capable of activating several cell types including leukocytes and endothelial cells. IL-1 induces and amplifies immunological responses by promoting the production and expression of adhesion molecules, cytokines, chemokines and other inflammatory mediators such as prostaglandin E₂ and nitric oxide (NO). As a consequence, local inflammation is amplified and sustained. In addition, the IL-1-induced production of inflammatory mediators results in fever, headache, hypotension and weight loss. Furthermore, IL-1 is a hematopoietic growth factor and has been shown to reduce the nadir of leukocytes and platelets in patients during bone marrow transplantation. IL-1 has also been shown to promote angiogenesis by inducing the production of vascular endothelial growth factor, thereby promoting pannus formation and blood supply in rheumatic joints. IL-1 has been shown to promote the bone and cartilage degradation in rheumatic diseases. Lastly, IL-1 has been implicated as an important player in the inflammatory response in cardiovascular and fibrotic diseases.

### IL-33

IL-33 is normally released by damaged or necrotic barrier cells (endothelial and epithelial cells), acting as an alarmin, an endogenous danger signal, to alert the immune system of tissue damage during trauma or infection (Liew FY, Interleukin-33 in health and disease. Nature Reviews Immunology, 16, 676-689 (2016)). IL-33 induces T helper 2 (Th2) cells, mast cells, type 2 innate lymphoid cells, eosinophils, and basophils to produce type 2 cytokines (e.g. IL-5, IL-13). IL-33 is also known to target endothelial cells and induce angiogenesis. As a Th2 inducing cytokine, IL-33 has been implicated in e.g. asthma, allergic diseases, inflammatory bowel disease, and dermatitis. IL-33 can potently stimulate a wide range of cells and its pleiotropic nature is reflected in the role of IL-33 in tissue and metabolic homeostasis, infection, inflammation, cancer and diseases of the central nervous system.

### IL-36

The IL-36 cytokines α, β, γ are expressed in a variety of cell types, with abundant expression in e.g. keratinocytes, bronchial epithelium, neuronal cells, dendritic cells, and macrophages. IL-36 is most active in barrier tissues (like the skin, lung, and intestines), suggesting that their main responsibility is to regulate the interaction of the environment and the body. IL-36 is known to activate NF-κB and mitogen-activated protein kinases in target cells expressing the IL-36 receptor, such as keratinocytes, monocytes, dendritic cells and CD4 T cells. Emerging evidence indicates that IL-36 signaling is involved in the activation of innate and adaptive immune responses (Ding L, IL-36 cytokines in autoimmunity and inflammatory disease, Oncotarget, Vol. 9, (No. 2), pp: 2895-2901 (2018)).

Besides its critical role in inflammatory skin diseases such as psoriasis and atopic dermatitis, emerging evidence suggests that aberrant IL-36 activities also promote inflammatory diseases in the lung, kidneys, and intestines, underscoring the potential of IL-36 as a therapeutic target for common inflammatory diseases.

Interestingly, all cytokines described herein (IL-1α, IL-1β, IL-33, IL-36α, IL-36β, IL-36γ) have also been shown to target stromal cells such as fibroblasts and endothelial cells. Consequently, apart from the well-established impact of these cytokines in inflammatory diseases and disorders, these cytokines are also implicated in fibrotic diseases or disorders.

The antibody or antigen-binding fragment thereof, according to the first aspect of the invention, has the capacity to inhibit signaling of the interleukin-1 (IL-1) family of cytokine ligands and/or receptors. The person skilled in the art will appreciate that inhibition occurs upon binding of the antibody or antigen-binding fragment to the epitope on IL1RAP.

In some embodiments, the antibody or antigen-binding fragment thereof with binding specificity for IL1RAP is capable of inhibiting signaling upon binding to IL1RAP.

The person skilled in the art will appreciate that binding of the antibody or antigen-binding fragment thereof to IL1RAP can affect IL1RAP in different ways, for example on a molecular level or on a conformational level. However, the person skilled in the art will understand that it today might not be known completely how binding of the antibody or antigen-binding fragment thereof to IL1RAP affects IL1RAP. One possibility is that binding of the antibody or antigen-binding fragment thereof to IL1RAP can affect the association of IL1RAP with IL-1 receptor, IL-33 receptor and/or IL-36 receptor, respectively. Consequently, the appropriate receptor complex consisting of IL1RAP as co-receptor and any one of IL-1 receptor, IL-33 receptor or IL-36 receptor, might not be formed. Consequently, when cytokines such as IL-1α, IL-1β, IL-33, IL-36α, IL-36β and IL-36γ bind to their cognate receptor (IL-1 receptor, IL-33 receptor or IL-36 receptor, respectively), signaling of these cytokines might be impaired, such as inhibited, such as blocked, such as essentially completely inhibited, such as partially inhibited.

The person skilled in the art will appreciate that not all IL1RAP binding antibodies, binding to any one epitope of IL1RAP, can disturb the association between IL1RAP with IL-1 receptor, IL-33 receptor and/or IL-36 receptor, respectively, and thereby inhibit signaling. It is an achievement of the present invention to provide such an antibody.

In some embodiments, the antibody or antigen-binding fragment thereof with binding specificity for IL1RAP is capable of inhibiting signaling of interleukin-1 (IL-1) family cytokine ligands and/or receptors.

In some embodiments, the antibody or antigen-binding fragment thereof with binding specificity for IL1RAP is capable of inhibiting signaling of IL1RAP-dependent interleukin-1 (IL-1) family of cytokine ligands and/or receptors.

In some embodiments, the antibody or antigen-binding fragment thereof with binding specificity for IL1RAP is capable of inhibiting signaling of at least one cytokine selected from the group consisting of IL-1α, IL-1β, IL-33, IL-36α, IL-36β and IL-36γ, or any combination thereof. In some embodiments, the antibody or antigen-binding fragment thereof with binding specificity for IL1RAP thereof is capable of inhibiting signaling of IL-la, IL-1β, IL-33, IL-36α, IL-36β and IL-36γ. In some embodiments, the antibody or antigen-binding fragment thereof with binding specificity for IL1RAP is capable of inhibiting signaling of IL-1α. In some embodiments, the antibody or antigen-binding fragment thereof with binding specificity for IL1RAP is capable of inhibiting signaling of IL-1β. In some embodiments, the antibody or antigen-binding fragment thereof with binding specificity for IL1RAP is capable of inhibiting signaling of IL-33. In some embodiments, the antibody or antigen-binding fragment thereof with binding specificity for IL1RAP thereof is capable of inhibiting signaling of IL-36α. In some embodiments, the antibody or antigen-binding fragment thereof with binding specificity for IL1RAP is capable of inhibiting signaling of IL-36β. In some embodiments, the antibody or antigen-binding fragment thereof with binding specificity for IL1RAP is capable of inhibiting signaling of IL-36γ.

The person skilled in the art will appreciate that inhibition of signaling can be of a varying degree. Inhibition of signaling can be complete or essentially complete, such as blocking of signaling. Inhibition of signaling can also be partial, such as decreasing the signaling, such as non-complete. “Essentially complete” is to be understood as “complete” given uncertainties of assessing completeness associated with the methodology used to measure the inhibition of signaling.

For example, signaling may be inhibited by at least 10%, 20%, 30%, 50%, 60%, 75%, 80%, 85%, 90%, 95%, 98%, 99% or more relative to signaling in the absence of the antibody or antigen-binding fragment of the invention. In some embodiments, inhibition of signaling is between 10 to 100% relative to signaling in the absence of the antibody or antigen-binding fragment of the invention. More preferably, the inhibition of signaling is between 25 to 100%. Even more preferably, the inhibition of signaling is between 50 to 100%. Signaling may be inhibited by 100% relative to signaling in the absence of the antibody or antigen-binding fragment of the invention.

The degree of inhibition of IL-1, IL-33 and/or IL-36 signaling by the antibody or antigen-binding fragment of the invention may be determined using methods well known in the art, for example by the method used in Example 2.

In preferred embodiments, the antibody or antigen-binding fragment thereof with binding specificity for IL1RAP is capable of essentially completely inhibiting signaling. In preferred embodiments, inhibiting signaling is essentially complete inhibition of signaling. In preferred embodiments, the antibody or antigen-binding fragment thereof with binding specificity for IL1RAP is capable of essentially completely inhibiting signaling of at least one cytokine selected from the group consisting of IL-1α, IL-1β, IL-33, IL-36α, IL-36β and IL-36γ, or any combination thereof. In preferred embodiments, the antibody or antigen-binding fragment thereof with binding specificity for IL1RAP is capable of essentially completely inhibiting signaling of IL-1α, IL-1β, IL-33, IL-36α, IL-36β and IL-36γ.

The antibody might further inhibit signaling to a certain degree, meaning not essentially completely, meaning partially. Consequently, inhibition of signaling can be not essentially complete inhibition, meaning partial inhibition. In some embodiments, the antibody or antigen-binding fragment thereof with binding specificity for IL1RAP is capable of partially inhibiting signaling. In some embodiments, inhibiting signaling is partial inhibition of signaling. In some embodiments, the antibody or antigen-binding fragment thereof with binding specificity for IL1RAP is capable of partially inhibiting signaling of at least one cytokine selected from the group consisting of IL-1α, IL-1β, IL-33, IL-36α, IL-36β and IL-36γ, or any combination thereof. In some embodiments, the antibody or antigen-binding fragment thereof with binding specificity for IL1RAP is capable of partially inhibiting signaling of IL-1α, IL-1β, IL-33, IL-36α, IL-36β and IL-36γ.

In some embodiments, the antibody or antigen-binding fragment thereof with binding specificity for IL1RAP is capable of inhibiting signaling of IL-1α, IL-1β, IL-33, IL-36α, IL-36β and/or IL-36γ by at least 10%, 20%, 30%, 50%, 60%, 75%, 80%, 85%, 90%, 95%, 98%, 99% relative to signaling in the absence of the antibody or antigen-binding fragment of the invention.

The person skilled in the art will appreciate that complete inhibition is desirable in some situations where a process, e.g. a disease process depending on signaling of IL-1α, IL-1B, IL-33, IL-36α, IL-36β and/or IL-36γ, should be affected completely and rapidly to alleviate or treat the disease. In other situations, partial inhibition is desirable where a process, e.g. a disease process depending on signaling of IL-1α, IL-1β, IL-33, IL-36α, IL-36β and/or IL-36γ, should be modified but not be affected completely, for example to avoid side effects of complete inhibition. The person skilled in the art will further appreciate that biological systems are complex and entail various known or unknown feedback loops, which can make it challenging to assess if a process is inhibited completely. Further, this assessment also depends on the sensitivity of the method with which the process, e.g. signaling, is measured. The person skilled in the art will know which method and cut-off values are established and accepted in the field to use to assess the completeness or essential completeness of inhibition of signaling or to assess the degree of partial inhibition of signaling.

As shown and explained in detail in the Examples below, antibodies can be subjected to modifications to optimize certain properties.

In some embodiments, the antibody or antigen-binding fragment thereof with binding specificity for IL1RAP is humanized. In some embodiments, the antibody or antigen-binding fragment thereof with binding specificity for IL1RAP is a human antibody. In some embodiments, the antibody or antigen-binding fragment thereof with binding specificity for IL1RAP is de-immunized. In some embodiments, the antibody or antigen-binding fragment thereof with binding specificity for IL1RAP is humanized and de-immunized. In some embodiments, the antibody or antigen-binding fragment thereof is monoclonal.

### Properties of the Antibody

The antibodies of the present invention were identified after extensive screening of a large number of anti-IL1RAP antibodies, on the basis of exhibiting properties that make them particularly suitable as diagnostic and therapeutic agents for inflammatory, fibrotic and/or neoplastic diseases or disorders.

Thus, in some embodiments, the antibody or antigen-binding fragment thereof with binding specificity for IL1RAP exhibits one or more of the following properties:
a) a binding affinity (K_{D}) for IL1RAP characterized by a K_{D}-value of 3 nM or less;
b) binding to domain 3 of IL1RAP;
c) binding to aa239 to 251, 263 to 277, 292 to 306, and/or 323 to 343 of IL1RAP:
d) cross-reactivity with IL1RAP from mouse, rat, cynomolgus monkey, dog and/or pig;
e) an inhibitory action on IL-1α signaling;
f) an inhibitory action on IL-1β signaling;
g) an inhibitory action on IL-33 signaling;
h) an inhibitory action on IL-36α signaling;
i) an inhibitory action on IL-36β signaling;
j) an inhibitory action on IL-36γ signaling;
k) ADCC of IL1RAP-expressing cells.

In some embodiments, the antibody or antigen-binding fragment thereof with binding specificity for IL1RAP exhibits one, two, three, four, five, six, seven, eight, nine or all of the above listed properties.

In other embodiments, the antibody or antigen-binding fragment thereof with binding specificity for IL1RAP exhibits all of the following properties:
a) a binding affinity (K_{D}) for IL1RAP characterized by a Ko-value of 3 nM or less;
b) binding to domain 3 of IL1RAP;
c) binding to aa239 to 251, 263 to 277, 292 to 306, and/or 323 to 343 of IL1RAP;
d) cross-reactivity with IL1RAP from mouse, rat, cynomolgus monkey, dog and/or pig;
e) an inhibitory action on IL-1α signaling;
f) an inhibitory action on IL-1β signaling;
g) an inhibitory action on IL-33 signaling;
h) an inhibitory action on IL-36α signaling;
i) an inhibitory action on IL-36β signaling;
j) an inhibitory action on IL-36γ signaling;
k) ADCC of IL1RAP-expressing cells.

In some embodiments, the binding affinity (K_{D}) of the antibody or antigen-binding fragment thereof for IL1RAP is 3 nM or less, such as 2.75 nM, for example 2.5 nM, such as 2 nM, for example 1.75 nM, such as 1.5 nM, for example 1.25 nM, such as 1 nM, for example 0.75 nM, such as 0.5 nM or for example 0.5 nM.

In some embodiments, the binding affinity (K_{D}) of the antibody or antigen-binding fragment thereof for IL1RAP is 3000 pM or less, such as 2750 pM, for example 2500 pM, such as 2000 pM, for example 1750 pM, such as 1500 pM, for example 1250 pM, such as 1000 pM, for example 750 pM, such as 700 pM, for example 650 pM, such as 600 pM, for example 550 pM, such as 500 pM, for example 450 pM, such as 400 pM, for example 350 pM, such as 300 pM, for example 250 pM, such as 200 pM, for example 150 pM, such as 100 pM, for example 50 pM.

The person skilled in the art will appreciate that the determination of the binding affinity of an antibody or antigen-binding fragment depends on the method for determining that binding affinity.

The person skilled in the art will appreciate that an IL1RAP antibody, apart from being able to inhibit cytokine signaling (for example of IL-1α, IL-1β, IL-33, IL-36α, IL-36β, and/or IL-36γ), can exhibit one or more further functions. These functions can be conveyed by the constant region of the antibody or the Fc region. Examples of these functions are antibody-dependent cell-mediated cytotoxicity (ADCC) and/or antibody-dependent cellular phagocytosis (ADCP), thereby leading to the killing of target cells, such as IL1RAP-expressing tumour cells.

In some embodiments, the antibody or antigen-binding fragment thereof with binding specificity for IL1RAP is capable of inducing ADCC of cells expressing IL1RAP. In some embodiments, the antibody or antigen-binding fragment thereof with binding specificity for IL1RAP is not capable of inducing ADCC of cells expressing IL1RAP. In some embodiments, the antibody or antigen-binding fragment thereof with binding specificity for IL1RAP is capable of inducing ADCP of cells expressing IL1RAP. In some embodiments, the antibody or antigen-binding fragment thereof with binding specificity for IL1RAP is not capable of inducing ADCP of cells expressing IL1RAP. In some embodiments, the antibody or antigen-binding fragment thereof with binding specificity for IL1RAP is capable of inducing ADCC and ADCP of cells expressing IL1RAP.

### Modifications of the Antibody

In some embodiments, the antibody or antigen-binding fragment thereof with binding specificity for IL1RAP further comprises a moiety for increasing the in vivo half-life of the agent. In some embodiments, the antibody or antigen-binding fragment thereof with binding specificity for IL1RAP further comprises a moiety for increasing the in vivo half-life of the agent, wherein the moiety for increasing the in vivo half-life is selected from the group consisting of polyethylene glycol (PEG), human serum albumin, glycosylation groups, fatty acids and dextran.

In some embodiments, the antibody or antigen-binding fragment thereof with binding specificity for IL1RAP is PEGylated.

In some embodiments, the antibody or antigen-binding fragment thereof with binding specificity for IL1RAP fragment is covalently bound, directly or indirectly (e.g. via a chelator), to a functional moiety such as a cytotoxic or detectable moiety.

In some embodiments, the antibody or antigen-binding fragment thereof with binding specificity for IL1RAP comprises a cytotoxic moiety. In some embodiments, the antibody or antigen-binding fragment thereof with binding specificity for IL1RAP comprising a cytotoxic moiety, wherein the cytotoxic moiety comprises or consists of a radioisotope. In some embodiments, the antibody or antigen-binding fragment thereof with binding specificity for IL1RAP comprising a cytotoxic moiety which comprises or consists of a radioisotope wherein the radioisotope is selected from the group consisting of beta-emitters, auger-emitters, conversion electron-emitters, alpha-emitters, and low photon energy-emitters.

In some embodiments, the antibody or antigen-binding fragment thereof with binding specificity for IL1RAP comprising a cytotoxic moiety which comprises or consists of a radioisotope, wherein the radioisotope has an emission pattern of locally absorbed energy that creates a high dose absorbance in the vicinity of the agent. In some embodiments, the antibody or antigen-binding fragment thereof with binding specificity for IL1RAP comprising a cytotoxic moiety which comprises or consists of a radioisotope, wherein the radioisotope is selected from the group consisting of long-range beta-emitters, such as ⁹⁰Y, ³²P, ¹⁸⁶Re/¹⁸⁶Re; ¹⁶⁶Ho, ⁷⁶As/⁷⁷As, ¹⁵³Sm; medium range beta-emitters, such as ¹³¹I, ¹⁷⁷Lu, ⁶⁷Cu, ¹⁶¹Tb; low-energy beta-emitters, such as ⁴⁵Ca, ³⁵S or ¹⁴C; conversion or auger-emitters, such as ⁵¹Cr, ⁶⁷Ga, ⁹⁹Tc^{m}, ¹¹¹I, ¹²³I, ¹²⁵I, ²⁰¹Tl; and alpha-emitters, such as ²¹²Bi, ²¹³Bi, ²²³Ac, and ²²¹At. In some embodiments, the antibody or antigen-binding fragment thereof with binding specificity for IL1RAP comprising a cytotoxic moiety which comprises or consists of a radioisotope, wherein the radioisotope is ¹⁷⁷Lu.

In some embodiments, the antibody or antigen-binding fragment thereof with binding specificity for IL1RAP comprises a cytotoxic moiety, wherein the cytotoxic moiety comprises or consists of a cytotoxic drug. In some embodiments, the antibody or antigen-binding fragment thereof with binding specificity for IL1RAP comprising a cytotoxic moiety which comprises or consists of a cytotoxic drug, wherein the cytotoxic drug is selected from the group consisting of a cytostatic drug; an anti-androgen drug; cortisone and derivatives thereof; a phosphonate; a testosterone-5-α-reductase inhibitor; a boron addend; a cytokine; thapsigargin and its metabolites; a toxin (such as saporin or calicheamicin); a chemotherapeutic agent (such as an antimetabolite); or any other cytotoxic drug useful in the treatment of neoplastic disorders. In some embodiments, the antibody or antigen-binding fragment thereof with binding specificity for IL1RAP comprising a cytotoxic moiety which comprises or consists of a cytotoxic drug, wherein the cytotoxic drug is suitable for use in activation therapy, such as photon activation therapy, neutron activation therapy, neutron induced Auger electron therapy, synchrotron irradiation therapy, or low energy X-ray photon activation therapy.

In some embodiments, the antibody or antigen-binding fragment thereof with binding specificity for IL1RAP comprises a detectable moiety. In some embodiments, the antibody or antigen-binding fragment thereof with binding specificity for IL1RAP comprises a detectable moiety, wherein the detectable moiety comprises or consists of a radioisotope. In some embodiments, the antibody or antigen-binding fragment thereof with binding specificity for IL1RAP comprises a detectable moiety comprising or consisting of a radioisotope, wherein the radioisotope is selected from the group consisting of ^{99m}Tc, ¹¹¹In, ⁶⁷Ga, ⁶⁸Ga, ⁷²As, ⁸⁹Zr, ¹²³I and ²⁰¹Tl. In some embodiments, the antibody or antigen-binding fragment thereof with binding specificity for IL1RAP comprises a detectable moiety comprising or consisting of a radioisotope, wherein the radioisotope is ⁸⁹Zr.

In some embodiments, the antibody or antigen-binding fragment thereof with binding specificity for IL1RAP comprises a pair of detectable and cytotoxic radioisotopes, such as ⁸⁶Y/⁹⁰Y or ¹²⁴I/²¹¹At.

In some embodiments, the antibody or antigen-binding fragment thereof with binding specificity for IL1RAP comprises a radioisotope which is capable of simultaneously acting in a multi-modal manner as a detectable moiety and as a cytotoxic moiety.

In some embodiments, the antibody or antigen-binding fragment thereof with binding specificity for IL1RAP comprises a detectable moiety which comprises or consists of a paramagnetic isotope. In some embodiments, the antibody or antigen-binding fragment thereof with binding specificity for IL1RAP comprises a detectable moiety which comprises or consists of a paramagnetic isotope, wherein the paramagnetic isotope is selected from the group consisting of ¹⁵⁷Gd, ⁵⁵Mn, ¹⁶²Dy ⁵²Cr and ⁵⁶Fe.

In some embodiments, the antibody or antigen-binding fragment thereof with binding specificity for IL1RAP comprises a detectable moiety, wherein the detectable moiety is detectable by an imaging technique such as SPECT, PET, MRI, optical or ultrasound imaging.

In some embodiments, the antibody or antigen-binding fragment thereof with binding specificity for IL1RAP comprises a cytotoxic moiety and/or detectable moiety, wherein the cytotoxic moiety and/or detectable moiety is joined to the antibody or antigen-binding fragment thereof indirectly, via a linking moiety. In some embodiments, the linking moiety is a chelator. In some embodiments, the chelator is selected from the group consisting of derivatives of 1,4,7,10-tetraazacyclododecane-1,4,7,10,tetraacetic acid (DOTA), deferoxamine (DFO), derivatives of diethylenetriaminepentaacetic acid (DTPA), derivatives of S-2-(4-Isothiocyanatobenzyl)-1,4,7-triazacyclononane-1,4,7-triacetic acid (NOTA) and derivatives of 1,4,8,11-tetraazacyclododecan-1,4,8,11-tetraacetic acid (TETA).

In some embodiments, the antibody or antigen-binding fragment thereof with binding specificity for IL1RAP does not comprise a cytotoxic moiety or a detectable moiety.

The invention further includes an antibody or antigen-binding fragment thereof with binding specificity for human interleukin-1 receptor accessory protein (IL1RAP), wherein the antibody or antigen-binding fragment is capable of inhibiting the binding of the herein disclosed antibody 3G5, 43F5 and/or 48A5 to IL1RAP.

As used herein, the term “capable of inhibiting the binding of antibody 3G5, 43F5 and/or 48A5 to IL1RAP” means that the presence of another antibody inhibits, in whole or in part, the binding of 3G5, 43F5 and/or 48A5 to IL1RAP. Such competitive binding inhibition can be determined using assays and methods well known in the art, for example using BIAcore chips with immobilised IL1RAP and incubating with the 3G5, 43F5 and/or 48A5 with and without an antibody to be tested. Alternatively, a pair-wise mapping approach can be used, in which the antibody 3G5, 43F5 and/or 48A5 is immobilised to the surface of the BIAcore chip, IL1RAP antigen is bound to the immobilised antibody, and then a second antibody is tested for simultaneous IL1RAP-binding ability (see ‘BIAcore Assay Handbook’, GE Healthcare Life Sciences, 29-0194-00 AA 05/2012; the disclosures of which are incorporated herein by reference).

In a further alternative, competitive binding inhibition can be determined using flow cytometry. For example, to test whether a test antibody is able to inhibit the binding of the 3G5, 43F5 and/or 48A5 antibody to a cell surface antigen, cells expressing the antigen can be pre-incubated with the test antibody for 20 min before cells are washed and incubated with the 3G5, 43F5 and/or 48A5 antibody conjugated to a fluorophore, which can be detected by flow cytometry. If the pre-incubation with the test antibody reduces the detection of the 3G5, 43F5 and/or 48A5 antibody in flow cytometry, the test antibody inhibits the binding of the reference antibody to the cell surface antigen. If the antibody to be tested exhibits high affinity for IL1RAP, then a reduced pre-incubation period may be used (or even no pre-incubation at all).

### Production of the Antibody

A second aspect of the invention relates to a polynucleotide encoding the antibody or antigen-binding fragment of the first aspect of the invention, or a component polypeptide chain thereof.

The term “polynucleotide” as used herein includes DNA (e.g. genomic DNA or complementary DNA) and mRNA molecules, which may be single- or double-stranded.

In some embodiments, the polynucleotide is an isolated polynucleotide. In some embodiments, the polynucleotide is a cDNA molecule.

It will be appreciated by persons skilled in the art that the polynucleotide may be codon-optimised for expression of the antibody or antigen-binding fragment in a particular host cell, e.g. for expression in human cells (for example, see Angov, 2011, Biotechnol. J. 6(6):650-659, the disclosures of which are incorporated herein by reference).

In some embodiments, the polynucleotide encoding the antibody or antigen-binding fragment of the invention is encoding an antibody light chain or variable region thereof. In some embodiments, the polynucleotide encoding the antibody or antigen-binding fragment of the invention is encoding an antibody heavy chain or variable region thereof.

A third aspect of the invention relates to a vector comprising the polynucleotide according to the second aspect of the invention.

In some embodiments, the vector is an expression vector.

The term “expression vector” is defined herein as a DNA molecule, for example linear or circular, that comprises a polynucleotide encoding a polypeptide of the present invention (the antibody or antigen-binding fragment thereof) and is operably linked to additional nucleotides that provide for its expression. The terms “plasmid”, “expression vector” and “vector” are used interchangeably as the plasmid is generally the most commonly used form of vector at present. However, the invention is intended to include such other forms of expression vectors that serve equivalent functions that are known in the art. As used herein “expression vector” or “vector” refers to a DNA construct containing a DNA sequence that is operably linked to a suitable control sequence capable of effecting the expression of the DNA in a suitable host. Such control sequences may, e.g., include a promoter to effect transcription, an optional operator sequence to control such transcription, a sequence encoding suitable mRNA ribosome binding sites and sequences which control termination of transcription and translation. The vector may, e.g., be a plasmid, a phage or simply a potential genomic insert. Once transformed into a suitable host, the vector may, e.g., replicate and function independently of the host genome, or may, in some instances, integrate into the genome itself. Expression vectors are designed, for example, as described in Li et al. (Construction strategies for developing expression vectors for recombinant monoclonal antibody production in CHO cells, Mol Biol Rep. 2018 December; 45(6):2907-2912).

A fourth aspect of the invention relates to a recombinant host cell comprising the polynucleotide according to the second aspect of the invention or a vector according to the third aspect of the invention. The recombinant host cell may be a bacterial cell, a yeast cell, a mammalian cell, or a human cell.

A fifth aspect of the invention relates to a method for producing the antibody or antigen-binding fragment of the first aspect of the invention, the method comprising culturing the host cell of the fourth aspect of the invention comprising the polynucleotide of the second aspect of the invention or the vector of the third aspect of the invention, under conditions which permit expression of the encoded antibody or antigen-binding fragment thereof.

### Pharmaceutical Composition

A sixth aspect of the invention relates to a pharmaceutical composition comprising
the antibody or antigen-binding fragment of the first aspect of the invention,
the polynucleotide of the second aspect of the invention,
the vector of the third aspect of the invention, and/or
the host cell of the fourth aspect of the invention,
in a pharmaceutical composition, wherein the composition further comprises a pharmaceutically-acceptable diluent, carrier or excipient.

In some embodiments, the composition comprises an effective amount of
the antibody or antigen-binding fragment of the first aspect of the invention,
the polynucleotide of the second aspect of the invention,
the vector of the third aspect of the invention, and/or
the host cell of the fourth aspect of the invention.

It will be appreciated by persons skilled in the art that additional compounds may also be included in the pharmaceutical compositions, including, chelating agents such as EDTA, citrate, EGTA or glutathione.

The pharmaceutical compositions may be prepared in a manner known in the art that is sufficiently storage stable and suitable for administration to humans and animals. For example, the pharmaceutical compositions may be lyophilised, e.g. through freeze drying, spray drying, spray cooling, or through use of particle formation from supercritical particle formation.

The term “pharmaceutically acceptable” is intended to mean a non-toxic material that does not decrease the effectiveness of the IL1RAP-binding activity of the antibody or antigen-binding fragment of the invention. Pharmaceutically acceptable buffers, carriers, diluents or excipients, for example, are well-known in the art.

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelactic acid, PIPES, SSC, SSPE, POPSO, TAPS, TABS, TAPSO and TES.

The term “diluent” is intended to mean an aqueous or non-aqueous solution with the purpose of diluting the antibody or antigen-binding fragment in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the antibody or antigen-binding fragment of the invention. The adjuvant may be one or more of zinc, copper or silver salts with different anions, for example, but not limited to fluoride, chloride, bromide, iodide, thiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g. for facilitating lyophilisation.

Examples of polymers are starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone, all of different molecular weight, which are added to the composition, e.g., for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The antibody or antigen-binding fragment of the invention may be formulated into any type of pharmaceutical composition known in the art to be suitable for the delivery thereof.

In some embodiments, the pharmaceutical compositions of the invention may be in the form of a liposome, in which the antibody or antigen-binding fragment is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations can be found in for example U.S. Pat. No. 4,235,871, the disclosures of which are incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303, the disclosures of which are incorporated herein by reference.

In a further embodiment, the pharmaceutical compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polyvinyl imidazole, polysulphonate, polyethyleneglycol/polyethylene oxide, polyethyleneoxide/polypropylene oxide copolymers, polyvinylalcohol/polyvinylacetate of different degree of hydrolysis, and polyvinylpyrrolidone are used for thickening of the solution containing the agent. The polymers may also comprise gelatin or collagen.

Alternatively, the antibody or antigen-binding fragment may simply be dissolved in saline, water, polyethylene glycol, propylene glycol, ethanol or oils (such as safflower oil, corn oil, peanut oil, cottonseed oil or sesame oil), tragacanth gum, and/or various buffers.

It will be appreciated that the pharmaceutical compositions of the invention may include ions and a defined pH for potentiation of action of the active antibody or antigen-binding fragment. Additionally, the compositions may be subjected to conventional pharmaceutical operations such as sterilisation and/or may contain conventional adjuvants such as preservatives, stabilisers, wetting agents, emulsifiers, buffers, fillers, etc.

The pharmaceutical compositions according to the invention may be administered via any suitable route known to those skilled in the art. Thus, possible routes of administration include parenteral (intravenous, subcutaneous, and intramuscular), topical, ocular, nasal, pulmonar, buccal, oral, parenteral, vaginal and rectal. Also, administration from implants is possible.

In one preferred embodiment, the pharmaceutical compositions are administered parenterally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques.

Preferably, the pharmaceutical compositions are administered intravenously. They are conveniently used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Thus, the pharmaceutical compositions of the invention are particularly suitable for parenteral, e.g. intravenous, administration.

Alternatively, the pharmaceutical compositions may be administered intranasally or by inhalation (for example, in the form of an aerosol spray presentation from a pressurised container, pump, spray or nebuliser with the use of a suitable propellant, such as dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas). In the case of a pressurised aerosol, the dosage unit may be determined by providing a valve to deliver a metered amount. The pressurised container, pump, spray or nebuliser may contain a solution or suspension of the active antibody or antigen-binding fragment, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

Aerosol or dry powder formulations are preferably arranged so that each metered dose or ‘puff’ contains at least 1 mg of a compound of the invention for delivery to the patient. It will be appreciated that the overall daily dose with an aerosol will vary from patient to patient, and may be administered in a single dose or, more usually, in divided doses throughout the day.

Alternatively, the antibody or antigen-binding fragment of the invention can be administered in the form of a suppository or pessary, or they may be applied topically in the form of a lotion, solution, cream, ointment or dusting powder. The compounds of the invention may also be transdermally administered, for example, using a skin patch. They may also be administered by the ocular route.

For ophthalmic use, the antibody or antigen-binding fragment of the invention can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the antibody or antigen-binding fragment of the invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The pharmaceutical compositions will be administered to a patient in a pharmaceutically effective dose. A “therapeutically effective amount”, or “effective amount”, or “therapeutically effective”, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity.

Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals. Alternatively, the dose may be provided as a continuous infusion over a prolonged period.

In the context of diagnostic use of the antibody or antigen-binding fragment of the invention, a “pharmaceutically effective amount”, or “effective amount”, or “diagnostically effective”, as used herein, refers to that amount which provides a detectable signal for diagnosis, e.g. for in vivo imaging purposes.

The antibody or antigen-binding fragment can be formulated at various concentrations, depending on the efficacy/toxicity of the antibody or antigen-binding fragment being used. For example, the formulation may comprise the active antibody or antigen-binding fragment at a concentration of between 0.1 μM and 1 mM, more preferably between 1 μM and 500 μM, between 500 μM and 1 mM, between 300 μM and 700 μM, between 1 μM and 100 μM, between 100 μM and 200 μM, between 200 μM and 300 μM, between 300 μM and 400 μM, between 400 μM and 500 μM, between 500 μM and 600 μM, between 600 μM and 700 μM, between 800 μM and 900 μM or between 900 μM and 1 mM. Typically, the formulation comprises the active antibody or antigen-binding fragment at a concentration of between 300 μM and 700 μM.

Typically, the therapeutic dose of the antibody or antigen-binding fragment (with or without a therapeutic moiety) in a human patient will be in the range of 100 μg to 1 g per administration (based on a body weight of 70 kg, e.g. between 300 μg to 700 mg per administration). For example, the maximum therapeutic dose may be in the range of 0.1 to 10 mg/kg per administration, e.g. between 0.1 and 5 mg/kg or between 1 and 5 mg/kg or between 0.1 and 2 mg/kg. In some embodiments, the therapeutic dose may be 5, 20 or 50 mg/kg, or any range formed from these values. For example, the therapeutic dose may be from 5 to 50 mg/kg, from 5 to 20 mg/kg, or from 20 to 50 mg/kg. It will be appreciated that such a dose may be administered at different intervals, as determined by the oncologist/physician; for example, a dose may be administered daily, twice-weekly, weekly, bi-weekly or monthly.

It will be appreciated by persons skilled in the art that the pharmaceutical compositions of the invention may be administered alone or in combination with other therapeutic agents used in the treatment of inflammatory, fibrotic and/or neoplastic disorders or diseases.

In some embodiments, said composition is adapted for parenteral delivery, intravenous delivery, topical delivery, subcutaneous delivery, intramuscular delivery.

It will be further appreciated by persons skilled in the art that the antibody or antigen-binding fragment and pharmaceutical compositions or formulations of the present invention have utility in both the medical and veterinary fields. Thus, the methods of the invention may be used in the treatment of both human and non-human animals (such as horses, dogs and cats). Preferably, however, the patient is human.

### Indications and Use of the Antibody

A seventh aspect of the invention relates to
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention,
for use in medicine.

An eighth aspect of the invention relates to
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention,
for use in the prevention and/or treatment and/or alleviation and/or detection and/or diagnosis of a disease or disorder susceptible to treatment with an inhibitor of IL-1α, IL-1β, IL-33, IL-36α, IL-36β and/or IL-36γ signaling, optionally wherein the disease or disorder is associated with cells expressing IL1RAP.

In an alternative aspect to the eighth aspect of the invention, the invention relates to
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention,
for use in the prevention and/or treatment and/or alleviation and/or detection and/or diagnosis of a disease or disorder susceptible to treatment with an inhibitor of IL-1α, IL-1β, IL-33, IL-36α, IL-36β and/or IL-36γ signaling.

In another alternative aspect to the eighth aspect of the invention, the invention relates to the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention,
for use in the prevention and/or treatment and/or alleviation and/or detection and/or diagnosis of a disease or disorder, wherein the disease or disorder is associated with cells expressing IL1RAP.

In some embodiments, said disease or disorder is associated with IL-1α, IL-1β, IL-33, IL-36α, IL-36β and/or IL-36γ signaling. In some embodiments, said disease or disorder is associated with cells expressing IL1RAP.

By “treatment” we include both therapeutic and prophylactic or preventive treatment of the patient. The terms “preventive” or “prophylactic” are used to encompass the use of an antibody or antigen-binding fragment thereof, or formulation thereof, as described herein which either prevents or reduces the likelihood of an inflammatory disease or disorder, a fibrotic disease or disorder, a neoplastic disorder or disorder, which also includes the prevention or reduction of the spread, dissemination, or metastasis of neoplastic cells in a patient or subject. The term “prophylactic” also encompasses the use of an antibody or antigen-binding fragment thereof, or formulation thereof, as described herein to prevent recurrence of an inflammatory, fibrotic and/or neoplastic disease or disorder in a patient who has previously been treated for any of these diseases or disorders.

By “diagnosis” or “detection” we include the detection of cells which are associated with an inflammatory, fibrotic and/or neoplastic disease or disorder, either in vivo (i.e. within the body of a patient) or ex vivo (i.e. within a tissue or cell sample removed from the body of a patient).

By “alleviation” we mean, without being limited by it, decreased symptoms or processes associated with a disease or disorder, i.e. resulting in a milder disease or disorder.

By “an inflammatory, fibrotic and/or neoplastic disease or disorder associated with cells expressing IL1RAP” we include such diseases or disorders wherein the pathological cells which are responsible, directly or indirectly, for the disorder express IL1RAP on the cell surface. It will be appreciated that the cells expressing IL1RAP may be immune cells, cells of the connective tissue such as fibroblasts or neoplastic cells (cancer cells), e.g. tumour cells, per se. In addition, such cells include pathological stem cells (i.e. cancer stem cells, or CSCs) and progenitor cells which are responsible, directly or indirectly, for the development of an inflammatory, fibrotic and/or neoplastic disease or disorder in an individual. Examples of CSCs are disclosed in Visvader & Lindeman, 2008, Nat Rev Cancer 8:755-768, the disclosures of which are incorporated herein by reference.

Alternatively, or in addition, the cells expressing IL1RAP may be associated indirectly with an inflammatory, fibrotic and/or neoplastic disease or disorder, for example, they may mediate cellular processes required for the cells to survive. The antibody or antigen-binding fragment thereof of the invention may in this event target cells essential for the maintenance of inflammatory and/or fibrotic processes, or, for example, blood supply of the tumour (angiogenesis) or cells inhibiting a beneficial immune response directed against the malignant cells (e.g. suppressive macrophages or T cells).

Depending upon whether it is therapeutically desirable to kill the target cells expressing IL1RAP, for example in the case of neoplastic cells, an antibody or antigen-binding fragment according to the first aspect of the invention may be used that it capable of inducing ADCC. For example, where the target cells expressing IL1RAP are cancer cells (such as CML, AML, ALL, melanoma, lung cancer cells, etc), it may be advantageous for the antibody or antigen-binding fragment to be capable of inducing ADCC in order to eliminate such cells. However, it will be appreciated that a therapeutic benefit may also be achieved using an antibody or antigen-binding fragment that lacks ADCC activity, for example through inhibition of IL-1α, IL-1β, IL-33, IL-36α, IL-36β and/or IL-36γ signaling leading to reduced angiogenesis in the vicinity of a cancer or tumour. Similarly, in the case of inflammatory and/or fibrotic diseases or disorders, it might be beneficial to affect cellular behaviour but not to kill the cells.

Conditions or disease states susceptible to treatment with an inhibitor of IL-1 and/or which may constitute an inflammatory, fibrotic and/or neoplastic disease or disorder associated with cells expressing IL1RAP, are well known in the art (see Dinarello et al., 2012, Nature Reviews 11:633-652 and Dinarello, 2014, Mol. Med. 20(suppl. 1):S43-S58, the disclosures of which are incorporated herein by reference) and include, but are not limited to, the following:
Rheumatoid arthritis, all types of arthritis, psoriatic arthritis, all types of juvenile arthritis, including systemic onset juvenile idiopathic arthritis (SOJIA), osteoarthritis, familial cold auto-inflammatory syndrome (FCAS), Muckle-Wells disease, neonatal onset multi-system inflammatory disease (NOMID), familial Mediterranean fever (FMF), pyogenic arthritis pyoderma gangrenosum and acne (PAPA) syndrome, adult onset Still's disease, hyper IgD syndrome, type 2 diabetes mellitus, macrophage activation syndrome, TNF receptor-associated periodic syndrome, Blau disease, ankylosing spondylitis, Sweet's disease, lupus arthritis, Alzheimer's disease, psoriasis, asthma, allergy, atherosclerosis, sarcoidosis, atopic dermatitis, systemic lupus erythematosus, bullous pemphigoid, type I diabetes mellitus, chronic obstructive pulmonary disease (COPD), Helicobacter pylori gastritis, inflammatory bowel disease (including ulcerative colitis and Crohn's disease), hepatitis, hepatitis C, ischaemia-reperfusion injury, multiple sclerosis, Neisserial or pneumococcal meningitis, tuberculosis, Behcet's syndrome, septic shock, graft versus host disease, adult T cell leukaemia, multiple myeloma, periodontitis, obesity and obesity-related diseases (for example, metabolic syndrome, cardiomegaly, congestive heart failure, myocardial infarction, varicose veins, polycystic ovarian syndrome, gastroesophageal reflux disease (GERD), fatty liver disease, colorectal cancer, breast cancer, uterine cancer, chronic renal failure, stroke and hyperuricemia), intervertebral disc disease, irritable bowel syndrome, Schnitzler syndrome, allergy/atopic dermatitis, acne inversa, Behcet's disease, cardiac fibrosis, cardiovascular diseases, cryopyrin-associated periodic syndromes, cystic fibrosis, Goodpasture's syndrome, Guillain-Barre syndrome, kidney fibrosis, liver fibrosis, lung fibrosis (pulmonary fibrosis), skin fibrosis (dermal fibrosis), myocarditis, autoimmune myocarditis, organ dysfunction associated with organ transplantation, pancreatitis, peritonitis, uveitis, vasculitis, pneumonia, pulmonary hypertension, sclerodermatous chronic graft-versus-host disease, sepsis, Sjögren's syndrome, systemic sclerosis, Takayasu's arteritis, gout, endometriosis, primary biliary cholangitis (PBC), allergic rhinitis, myasthenia gravis, Grave's disease, pemphigus, celiac disease, dermatitis herpetiformis, discoid lupus erythematosus, dermatomyositis, IgG4-related disease, alopecia areata, palmoplantar pustulosis (PPP), ichthyosis, hidradenitis suppurativa (HS), and Kawasaki disease.

Blockade of IL-1 signaling is also believed to be beneficial in the treatment of myocardial infarction. An extensive clinical trial is sought to confirm the efficacy of anti-IL-1β antibody blockade (using Canakinumab) following myocardial infarction (the CANTOS trail; see Ridker et al., 2011, Am Heart Journal 162(4):597-605, the disclosures of which are incorporated herein by reference).

For such indications, it will be appreciated that a therapeutic benefit may also be achieved using an antibody or antigen-binding fragment that binds IL1RAP and thereby blocking IL-1 and/or IL-33 and/or IL-36 signaling associated with immune cells. Such antibody could be modified to lack ADCC activity.

In some embodiments, said disease or disorder is an inflammatory and/or fibrotic and/or neoplastic disease or disorder.

In relation to the therapeutic and prophylactic aspects of the invention, it will be appreciated by persons skilled in the art that binding of the antibody, or antigen-binding fragment thereof, to IL1RAP present on the surface of the cells associated with the inflammatory, fibrotic and/or neoplastic disease or disorder may lead to a modulation (i.e. an increase or decrease) of a biological activity of IL1RAP. However, such a modulatory effect is not essential; for example, the antibody or antigen-binding fragment thereof of the invention may elicit a therapeutic and prophylactic effect simply by virtue of binding to IL1RAP on the surface of the cells associated with the disease or disorder, which in turn may trigger the immune system to induce processes, such as cell death (e.g. by ADCC and/or by the presence within the agent of a cytotoxic/radioactive moiety).

In some embodiments, the antibody or antigen-binding fragment thereof with binding specificity for IL1RAP inhibits the biological activity of IL1RAP.

By “biological activity of IL1RAP” we include any interaction or signaling event which involves IL1RAP on the cells associated with the inflammatory, fibrotic and/or neoplastic disease or disorder. For example, in one embodiment the antibody or antigen-binding fragment thereof is capable of blocking binding of one or more co-receptors to IL1RAP (such as IL1R1, ST2, C-KIT and/or IL1RL2). Further, as elaborated above, in some embodiments, the antibody or antigen-binding fragment thereof with binding specificity for IL1RAP inhibits signaling of IL-1α, IL-1β, IL-33, IL-36α, IL-36β and/or IL-36γ.

The person skilled in the art will understand that “inhibiting signaling of cytokines” (such as cytokines of the IL-1 family, such as inhibiting signaling of IL-1α, IL-1β, IL-33, IL-36α, IL-36β and/or IL-36γ) leads to an inhibition of the biological activity of IL1RAP.

Such inhibition of the biological activity of IL1RAP by an antibody or antigen binding fragment thereof of the invention may be in whole or in part. For example, the antibody or antigen binding fragment thereof may inhibit the biological activity of IL1RAP by at least 10%, preferably at least 20%, 30%, 40%, 50%, 60%, 70%, 80% or 90%, and most preferably by 100% compared to the biological activity of IL1RAP in cells associated with the inflammatory, fibrotic and/or neoplastic disease or disorder which have not been exposed to the antibody or antigen-binding fragment thereof. In a preferred embodiment, the antibody or antigen-binding fragment thereof is capable of inhibiting the biological activity of IL1RAP by 50% or more compared to the biological activity of IL1RAP in cells associated with the inflammatory, fibrotic and/or neoplastic disease or disorder which have not been exposed to the antibody or antigen-binding fragment.

Likewise, it will be appreciated that inhibition of growth and/or proliferation of the cells associated with the neoplastic disease or disorder may be in whole or in part. For example, the antibody or antigen-binding fragment thereof may inhibit the growth and/or proliferation of the cells associated with the neoplastic disease or disorder by at least 10%, preferably at least 20%, 30%, 40%, 50%, 60%, 70%, 80% or 90%, and most preferably by 100% compared to the growth and/or proliferation of cells associated with the neoplastic disease or disorder which have not been exposed to the antibody or antigen binding fragment thereof.

### IL-1, IL-33 and IL-36 in Disease

IL-1 is implicated in a wide range of diseases and conditions ranging from gout to cancer (for reviews, see Dinarello et al., 2012, Nature Reviews 11:633-652 and Dinarello, 2014, Mol. Med. 20(suppl. 1):S43-S58; the disclosures of which are incorporated herein by reference), including:
Joint, bone and muscle diseases, such as rheumatoid arthritis and osteoarthritis;
Hereditary systemic autoinflammatory diseases, such as familial Mediterranean fever;
Systemic autoinflammatory diseases, such as systemic juvenile idiopathic arthritis and adult-onset Still's disease;
Common inflammatory diseases, such as gout and type 2 diabetes;
Acute-onset ischemic diseases, such as myocardial infarction; and
Cancer.

A number of therapies for blocking IL-1 activity are approved and in development. Targeting IL-1 began in 1993 with the introduction of anakinra (Kineret; Amgen), a recombinant form of the naturally occurring IL-1 receptor antagonist (IL-1Ra or IL1RA), which blocks the activity of both IL-1α and IL-1β; this therapeutic has since been used to demonstrate a role for IL-1 in numerous diseases (see above). Anakinra currently dominates the field of IL-1 therapeutics owing to its good safety record, short half-life and multiple routes of administration. Neutralising IL-1 with antibodies or soluble receptors has also proved to be effective, and the soluble decoy receptor rilonacept (Arcalyst; Regeneron) and the anti-IL-1β neutralizing monoclonal antibody canakinumab (Ilaris; Novartis) have now been approved. Other therapeutic approaches, including IL-1α neutralisation, a therapeutic vaccine targeting IL-1β and a chimeric IL-1Ra, are in early clinical trials. In addition, orally active small-molecule inhibitors of IL-1 production, such as caspase 1 inhibitors, have been developed and are being tested.

Similarly, as elaborated above, there is emerging evidence of the implication of IL-33 in disease. As elaborated above, IL-33 has been implicated in e.g. asthma, allergic diseases, inflammatory bowel disease, and dermatitis. Importantly, IL-33 is implicated in fibrosis and inflammation (Kotsiou et al. 2018, IL-33/ST2 Axis in Organ Fibrosis, Front Immunol. 2018 Oct. 24; 9:2432). IL-33 can potently stimulate a wide range of cells and its pleiotropic nature is reflected in the role of IL-33 in tissue and metabolic homeostasis, infection, inflammation, cancer and diseases of the central nervous system.

Similarly, as elaborated above, there is emerging evidence of the implication of IL-36 in disease, even though this field is still evolving and, compared to IL-1, the knowledge relating to IL-36 is still limited. As elaborated above, emerging evidence indicates that IL-36 signaling is involved in the activation of innate and adaptive immune responses (Ding L, IL-36 cytokines in autoimmunity and inflammatory disease, Oncotarget, Vol. 9, (No. 2), pp: 2895-2901 (2018)). Besides its role in inflammatory skin diseases such as psoriasis and atopic dermatitis, emerging evidence suggests that aberrant IL-36 activities also promote inflammatory diseases in the lung, kidneys, and intestines, underscoring the potential of IL-36 as a therapeutic target for common inflammatory diseases.

The person skilled in the art will appreciate that the antibody or antigen-binding fragment thereof of the present invention may have or is expected to have profound effects on inflammatory and/or fibrotic diseases or disorders, for example due to the ability to inhibit signaling of IL-1α, IL-1β, IL-33, IL-36α, IL-36β and/or IL-36γ, thereby targeting several IL-dependent pathways at the same time.

In some embodiments, said disease or disorder is an inflammatory and/or fibrotic disease or disorder. In some embodiments, said disease or disorder is an inflammatory and/or fibrotic disease or disorder is selected from the group consisting of rheumatoid arthritis, all types of arthritis, psoriatic arthritis, all types of juvenile arthritis, including systemic onset juvenile idiopathic arthritis (SOJIA), osteoarthritis, familial cold auto-inflammatory syndrome (FCAS), Muckle-Wells disease, neonatal onset multi-system inflammatory disease (NOMID), familial Mediterranean fever (FMF), pyogenic arthritis pyoderma gangrenosum and acne (PAPA) syndrome, adult onset Still's disease, hyper IgD syndrome, type 2 diabetes mellitus, macrophage activation syndrome, TNF receptor-associated periodic syndrome, Blau disease, ankylosing spondylitis, Sweet's disease, lupus arthritis, Alzheimer's disease, psoriasis, asthma, allergy, atherosclerosis, sarcoidosis, atopic dermatitis, systemic lupus erythematosus, bullous pemphigoid, type I diabetes mellitus, chronic obstructive pulmonary disease (COPD), Helicobacter pylori gastritis, inflammatory bowel disease (including ulcerative colitis and Crohn's disease), hepatitis, hepatitis C, ischaemia-reperfusion injury, multiple sclerosis, Neisserial or pneumococcal meningitis, tuberculosis, Behcet's syndrome, septic shock, graft versus host disease, adult T cell leukaemia, multiple myeloma, periodontitis, obesity and obesity-related diseases (for example, metabolic syndrome, cardiomegaly, congestive heart failure, myocardial infarction, varicose veins, polycystic ovarian syndrome, gastroesophageal reflux disease (GERD), fatty liver disease, colorectal cancer, breast cancer, uterine cancer, chronic renal failure, stroke and hyperuricemia), intervertebral disc disease, irritable bowel syndrome, Schnitzler syndrome, allergy/atopic dermatitis, acne inversa, Behcet's disease, cardiac fibrosis, cardiovascular diseases, cryopyrin-associated periodic syndromes, cystic fibrosis, Goodpasture's syndrome, Guillain-Barre syndrome, kidney fibrosis, liver fibrosis, lung fibrosis (pulmonary fibrosis), skin fibrosis (dermal fibrosis), myocarditis, autoimmune myocarditis, organ dysfunction associated with organ transplantation, pancreatitis, peritonitis, uveitis, vasculitis, pneumonia, pulmonary hypertension, sclerodermatous chronic graft-versus-host disease, sepsis, Sjögren's syndrome, systemic sclerosis, Takayasu's arteritis, gout, endometriosis, primary biliary cholangitis (PBC), allergic rhinitis, myasthenia gravis, Grave's disease, pemphigus, celiac disease, dermatitis herpetiformis, discoid lupus erythematosus, dermatomyositis, IgG4-related disease, alopecia areata, palmoplantar pustulosis (PPP), ichthyosis, hidradenitis suppurativa (HS), and Kawasaki disease.

In some embodiments, said disease or disorder is systemic sclerosis, also called scleroderma or systemic scleroderma. In some embodiments, said disease or disorder is peritonitis, such as acute peritonitis. In some embodiments, said disease or disorder is psoriasis. In some embodiments, said disease or disorder is psoriatic arthritis. In one embodiment said disease or disorder is psoriasis and psoriatic arthritis.

In some embodiments, said disease or disorder is atherosclerosis. In some embodiments, the use of the herein disclosed invention in the prevention, treatment, alleviation, detection and/or diagnosis of atherosclerosis comprises the reduction of atherosclerosis, for example the reduction of clinical signs, symptoms or pathophysiological correlates of atherosclerosis, for example reduction of atherosclerotic plaque inflammation, such as aortic plaque inflammation, and/or a reduction in plaque size (for example, plaque volume and/or plaque area).

In some embodiments, said disease or disorder is lung fibrosis, also known as pulmonary fibrosis.

Fibrosis is the hallmark of a multitude of diseases. In some cases, fibrosis may be an integral part of the disease, meaning that fibrosis may be the consequence of aberrant or pathologic cellular behaviour leading to a pathological deposition of extracellular matrix proteins. In other cases, fibrosis may be a secondary event occurring in diseased tissue, in parallel to the primary disease or in the course of the healing process. For example, fibrosis may be scar formation. Any type of fibrosis may be targeted, for example treated or prevented, by the antibodies as disclosed herein. Fibrosis may be categorized as uncontrolled or dysregulated scar tissue formation, which can be caused by excessive accumulation of extracellular matrix components, such as collagen. Accordingly, the use of the herein disclosed invention in the prevention, treatment, alleviation, detection and/or diagnosis of fibrosis may be upstream of the prevention, treatment, alleviation, detection and/or diagnosis of a disease for which fibrosis is a hallmark. Alternatively, or additionally, the use of the herein disclosed invention in the prevention, treatment, alleviation, detection and/or diagnosis of fibrosis may be downstream of a disease for which fibrosis is a secondary event in said disease.

In some embodiments, said disease or disorder is myocarditis, such as autoimmune myocarditis.

In some embodiments, the use of the herein disclosed invention in the prevention, treatment, alleviation, detection and/or diagnosis of myocarditis, such as autoimmune myocarditis, comprises the reduction of myocarditis, such as autoimmune myocarditis, for example the reduction of clinical signs, symptoms or pathophysiological correlates of myocarditis, such as autoimmune myocarditis, for example reduction of deterioration in cardiac function, reduction in inflammation and/or reduction fibrosis.

In some embodiments, said disease or disorder is graft-versus-host disease, including chronic graft-versus-host disease and sclerodermatous chronic graft-versus-host disease. In one embodiment, said disease or disorder is dermal fibrosis and/or pulmonary fibrosis in graft-versus-host disease. In a particular embodiment, the graft-versus-host disease is caused by a syngeneic transplantation. In an alternative embodiment, the graft-versus-host disease is caused by an allogeneic transplantation.

In some embodiments, said disease or disorder is an acute inflammatory disease or disorder. In some embodiments, said disease or disorder is a chronic inflammatory disease or disorder.

The person skilled in the art will appreciate that diseases or disorders are grouped in different ways. While a disease or disorder may not be explicitly labelled as an inflammatory or fibrotic disease, it may still have an inflammatory and/or fibrotic components, for example inflammatory and/or fibrotic processes which form part or contribute to the disease or disorder.

In some embodiments, said disease or disorder has an inflammatory and/or fibrotic component. In some embodiments, said disease or disorder is an autoimmune disease or disorder. In some embodiments, the antibody or antigen-binding fragment of the first aspect of the invention inhibits inflammatory processes. In some embodiments, the antibody or antigen-binding fragment of the first aspect of the invention inhibits fibrotic processes. In some embodiments, the antibody or antigen-binding fragment of the first aspect of the invention inhibits proliferative processes.

### IL1RAP as a Biomarker for Neoplastic Diseases or Disorders

A neoplasm, indicative of neoplastic diseases or disorders (i.e. cancer), is a type of abnormal and excessive growth (also called neoplasia) of tissue or cells, for example tumours.

Tumour biomarkers or biomarkers of neoplastic diseases or disorders are endogenous proteins or metabolites whose amounts or modifications are indicative of tumour state, progression characteristics, and response to therapies. They are present in tumour tissues or body fluids and encompass a wide variety of molecules, including transcription factors, cell surface receptors, and secreted proteins. Effective tumour markers are in great demand since they have the potential to reduce cancer mortality rates by facilitating diagnosis of cancers at early stages and by helping to individualize treatments. During the last decade, improved understanding of carcinogenesis and tumour progression has revealed a large number of potential tumour markers. It is predicted that even more will be discovered in the near future with the application of current technologies such as tissue microarrays, antibody arrays, and mass spectrometry.

Interleukin-1 receptor accessory protein (IL1RAP) has previously been identified as cell-surface biomarker associated with haematological neoplastic disorders such as chronic myeloid leukemia (CML), acute myeloid leukemia (AML) and myelodysplatic syndromes (MDS) (for example, see WO 2011/021014 to Cantargia A B, Järås et al., 2010, Proc Nat/Acad Sci USA 107(37):16280-5, Askmyr et al., 2013, Blood. 121(18):3709-13 and Barreyro et al., 2012, Blood 120(6):1290-8, the disclosures of which are incorporated herein by reference). More recently, the usefulness of IL1RAP as a diagnostic and therapeutic biomarker for solid tumours, such as melanomas, has also been revealed (see WO 2012/098407 to Cantargia A B, the disclosures of which are incorporated herein by reference).

Therefore, in some embodiments of the above aspects of the invention, said disease or disorder is a neoplastic disease or disorder.

The person skilled in the art will appreciate that the antibody of the present invention may have profound effects on neoplastic diseases or disorders, for example due to the ability to inhibit signaling of IL-1α, IL-1β, IL-33, IL-36α, IL-36β and IL-36γ, thereby targeting several IL-dependent pathways at the same time.

In some embodiments, said disease or disorder is a neoplastic disease or disorder, wherein the neoplastic disease or disorder is a hematologic disease or disorder or a solid tumour.

In some embodiments, said neoplastic disease or disorder is a hematologic disease, wherein the neoplastic hematologic disease or disorder is selected from the group consisting of chronic myeloid leukemia (CML), myeloproliferative disorders (MPD), myelodysplastic syndrome (MDS), acute lymphoblastic leukemia (ALL) and acute myeloid leukemia (AML).

In some embodiments, said neoplastic disease or disorder is a solid tumour, wherein the solid tumour is selected from the group consisting of prostate cancer, breast cancer, lung cancer, colon cancer, colorectal cancer, melanomas, bladder cancer, brain/CNS cancer, cancer of urinary organs, biliary tract cancer (also known as bile duct cancer), cervical cancer, oesophageal cancer, gastric cancer, head/neck cancer, kidney cancer, liver cancer, lymphomas, ovarian cancer, pancreatic cancer, sarcomas, skin cancer and uterus cancer.

The person skilled in the art will appreciate that the antibody of the present invention is expected to have profound effects on neoplastic diseases or disorders, for example due to the ability to inhibit signaling of IL-1α, IL-1β, IL-33, IL-36α, IL-36β and/or IL-36γ, thereby targeting several IL-dependent pathways at the same time.

The person of skill in the art would, for example in the light of the evidence stated above, understand that the antibody of the present invention could be used for the prevention, treatment, alleviation, detection and/or diagnosis of any disease or disorder implicated by activity of IL-1α, IL-1β, IL-33, IL-36α, IL-36β and/or IL-36γ.

A ninth aspect of the invention relates to the antibody or antigen-binding fragment of
the first aspect of the invention,
the polynucleotide of the second aspect of the invention,
the vector of the third aspect of the invention,
the host cell of the fourth aspect of the invention, and/or
the composition of the sixth aspect of the invention,
for use in inducing cell death and/or inhibiting the growth and/or proliferation of pathological cells associated with a neoplastic disorder in a subject, or stem cells or progenitor cells thereof, wherein the cells express IL1RAP.

A tenth aspect of the invention relates to the use of
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention,
in the preparation of a medicament for the prevention, treatment, alleviation, detection and/or diagnosis of a disease or disorder susceptible to treatment with an inhibitor of IL-1α, IL-1β, IL-33, IL-36α, IL-36β and/or IL-36γ signaling, optionally wherein the disease or disorder is associated with cells expressing IL1RAP.

An eleventh aspect of the invention relates to the use of
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention,
in the preparation of a medicament for the detection and/or diagnosis of a disease or disorder associated with cells expressing IL1RAP.

A twelfth aspect of the invention relates to a method for the prevention and/or treatment and/or alleviation and/or detection and/or diagnosis of a disease or disorder susceptible to treatment with an inhibitor of IL-1α, IL-1β, IL-33, IL-36α, IL-36β and/or IL-36γ signaling in a subject, optionally wherein the disease or disorder is associated with cells expressing IL1RAP, comprising the step of administering to the subject an effective amount of
the antibody or antigen-binding fragment of the first aspect of the invention,
the polynucleotide of the second aspect of the invention,
the vector of the third aspect of the invention,
the host cell of the fourth aspect of the invention, and/or
the composition of the sixth aspect of the invention.

A thirteenth aspect of the invention relates to an in vitro method for the detection of cells expressing IL1RAP in a subject, the method comprising:
(a) providing a sample of cells from a subject to be tested, such as biopsy tissue or blood sample;
(b) optionally, extracting and/or purifying the cells present in the sample; (c) contacting the antibody or antigen-binding fragment of the first aspect of the invention with cells present in the sample;
(d) determining whether the antibody or antigen-binding fragment thereof binds to the cells;
wherein the binding of the antibody or antigen-binding fragment thereof to the cells is indicative of the presence of a disease or disorder associated with cells expressing IL1RAP in the tissue of a subject.

A fourteenth aspect of the invention relates to an in vitro method for identifying a patient with a disease or disorder associated with cells expressing IL1RAP who would benefit from treatment with the antibody or antigen-binding fragment of the first aspect of the invention, the method comprising:
(a) providing a sample of cells, such as biopsy tissue or blood sample from a patient to be tested;
(b) optionally, extracting and/or purifying the cells present in the sample;
(c) contacting the antibody or antigen-binding fragment of the first aspect of the invention with cells present in the sample;
(d) determining whether the antibody or antigen-binding fragment thereof binds to the cells;
wherein the binding of the antibody or antigen-binding fragment thereof to cells expressing IL1RAP is indicative of a patient who would benefit from treatment with the antibody or antigen-binding fragment of the first aspect of the invention.

A fifteenth aspect of the invention relates to a method for treating a patient with a disease or disorder associated with cells expression IL1RAP, the method comprising:
a) selecting a patient identified as having a disease or disorder associated with cells expressing IL1RAP using a method according to the fourteenth aspect of the invention; and
b) administering to said patient a therapeutic agent effective in the treatment of said disease or disorder.

A sixteenth aspect of the invention relates to a method for the detection of cells expressing IL1RAP, the method comprising:
(a) contacting an antibody or antigen-binding fragment thereof according to the first aspect with cells to be analysed for their expression of IL1RAP;
(b) determining whether the antibody or antigen-binding fragment thereof binds to the cells;
   wherein the binding of the antibody or antigen-binding fragment thereof to the cells is indicative of the presence of a disease or disorder associated with cells expressing IL1RAP in the tissue of a subject.

In some embodiments, the method of the sixteenth aspect is an in vitro method. In some embodiments, the method of the sixteenth aspect is an in vivo method.

The person skilled in the art will understand that, if the method of the sixteenth aspect is applied, the antibody or antigen-binding fragment with binding specificity for IL1RAP is administered to a subject, for example according to the descriptions herein.

### EXAMPLES

### Example 1: Immunization with IL1RAP to Generate Anti-IL1RAP Antibodies

### Aim

This example illustrates the isolation of anti-IL1RAP antibodies from rabbits or mice immunized with IL1RAP.

### Material and Methods

Rabbits were immunized four times followed by a boost using a mixture of recombinantly expressed ectodomains of human and murine IL1RAP, 0.2 mg of antigen per injection. Immunizations were carried out by subscapular injections every third week. First immunization was performed in Freund's complete adjuvant and second through fourth immunizations in Freund's incomplete adjuvant. For boosting, half the antigen amount was injected subscapularly in Freund's incomplete adjuvant and the other half intravenously.

Immune response of each animal was measured by ELISA using blood serum approximately ten days after the third immunization. The specific antibody titer was measured by ELISA, coating plates with human and mouse IL1RAP separately for immune response comparison. Two to three weeks after the fourth immunization, the animals were boosted once, and spleens were collected four to five days after the boost. Spleens of test animals were homogenized, cells frozen and stored in liquid nitrogen. Monoclonal antibody sequences were isolated from immunized rabbits using the approach described in Kivi et al. (Kivi G, HybriFree: a robust and rapid method for the development of monoclonal antibodies from different host species. BMC Biotechnology, 16:2 (2016)).

Streptavidin-coated 96-well plates were coated with biotinylated human or murine IL1RAP, or a mixture of both, at 5 μg/ml. Ten thousand spleen cells were used for each panning. In total, 48 panning reactions were performed. After incubation for 45 minutes, wells were washed with PBS to remove unbound cells. RNA was isolated from the bound cells and VH and VL cDNA was synthesized and used for construction of a combinatorial VH-VL library in a human IgG expression plasmid format. Plasmid DNA was purified and transfected into CHO cells for production of chimeric rabbit/human IgG1 pools.

Supernatants of antibody pools were analyzed by ELISA 48 hours after transfection and positive pools were identified. Bacteria containing plasmid DNA from the positive pools were plated on LB-ampicillin solid medium and single colonies were isolated and grown in liquid medium. Plasmid DNA was purified from the liquid cultures and transfected into CHO cells for transient antibody production. Supernatants were analyzed by ELISA 48-72 hours after transfection.

Alternatively, mice were immunized with human IL1RAP and hybridomas generated. The supernatants from growing hybridomas were screened with ELISA where the specific antibody titer was measured by coating plates with human and mouse IL1RAP separately for comparison.

### Results

Antibodies binding to human and/or murine IL1RAP were identified.

Chimeric rabbit/human IgG1 ELISA IL1RAP positive clones were further analyzed by sequencing and based on their functional properties, for example the ability to inhibit IL-1, IL-33 or IL-36 signaling. Chimeric antibodies 43F5 and 48A5 were selected for further characterization and optimization (see following Examples). The light chain variable regions of these antibodies comprise light chain variable regions comprising or consisting of the amino acid sequences of SEQ ID NOs: 21 and 23. The heavy chain variable regions of these antibodies comprise heavy chain variable regions comprising or consisting of the amino acid sequences of SEQ ID NOs: 22 and 24. To create whole antibodies, the variable regions were combined with light chain constant regions comprising or consisting of the amino acid sequence of SEQ ID NO: 35, and with IgG1 heavy chain constant regions, comprising or consisting of the amino acid sequence of SEQ ID NO: 36 or SEQ ID NO: 37.

Chimeric mouse/human IgG1 ELISA IL1RAP positive clones were further analyzed by sequencing and based on their functional properties, for example the ability to inhibit IL-1, IL-33 or IL-36 signaling. Chimeric antibody 3G5 was selected for further characterization and optimization (see following Examples). The light chain variable region of this antibody comprises a light chain variable region comprising or consisting of the amino acid sequence of SEQ ID NO: 19. The heavy chain variable region of this antibody comprises a heavy chain variable region comprising or consisting of the amino acid sequence of SEQ ID NO: 20. To create a whole antibody, the variable region was combined with the light chain constant region comprising or consisting of the amino acid sequence of SEQ ID NO: 35, and with the IgG1 heavy chain constant region, comprising or consisting of the amino acid sequence of SEQ ID NO: 36 or SEQ ID NO: 37.

### Conclusions

The monoclonal anti-IL1RAP chimeric rabbit/human IgG1 antibodies 43F5 and 48A5, and the chimeric mouse/human IgG1 antibody 3G5, were isolated.

### Example 2: Characterization of Anti-IL1RAP Antibodies 43F5, 48A5 and 3G5

### Aim

This example illustrates the characterization of the selected anti-human IL1RAP antibodies, 43F5, 48A5 and 3G5, generated as described in Example 1, in terms of binding to IL1RAP-expressing cells, inhibition of IL-1, IL-33 and IL-36 signaling, affinity measurements, domain mapping and species cross-reactivity.

### Material and Methods

### Antibody Binding to IL1RAP-Expressing Cells

The malignant melanoma cell line SKMEL-5, which expresses IL1RAP on the cell surface, was used for the analysis. SKMEL-5 cells were cultured according to standard procedures. SKMEL-5 cells (1×10⁵) were blocked with human Fc block and stained with serial dilutions (0.2-200 nM) of 43F5, 48A5, 3G5 or isotype control antibody. Following secondary staining using Alexa488-conjugated anti-human IgG antibody, samples were analyzed on a CytoFlex (Beckman Coulter). SKMEL-5 IL1RAP KO cells, stained with 43F5, 48A5, 3G5 or isotype control antibody (all at 200 nM) were used as negative controls.

### Inhibition of Cytokine Signaling

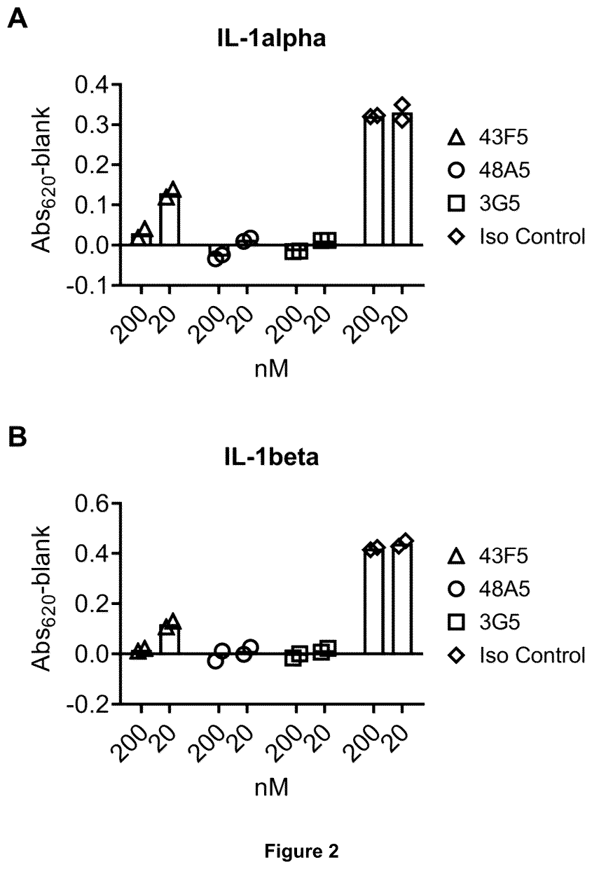

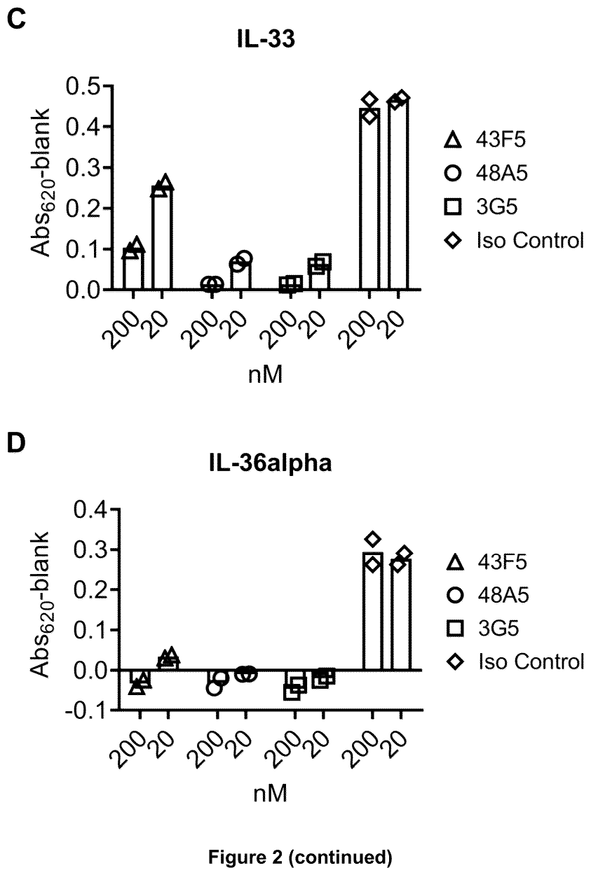

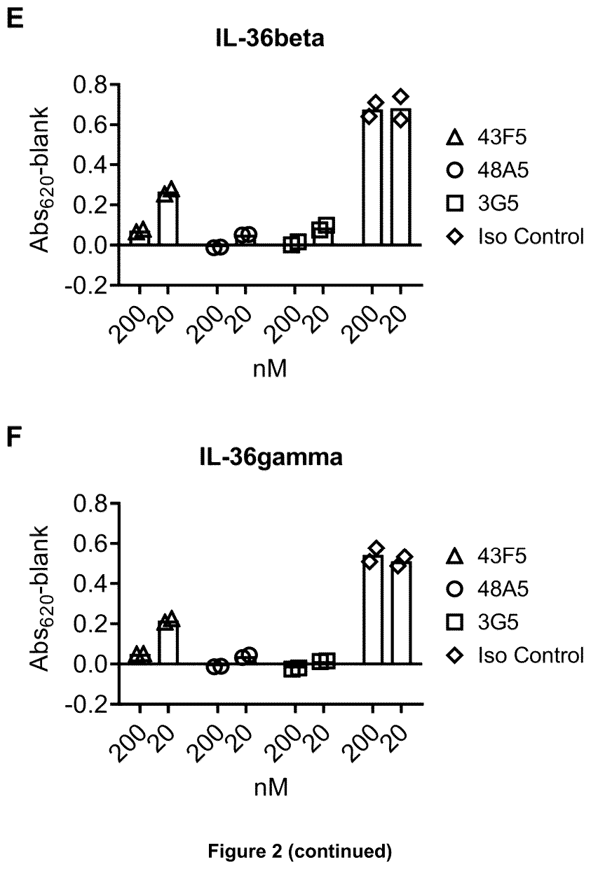

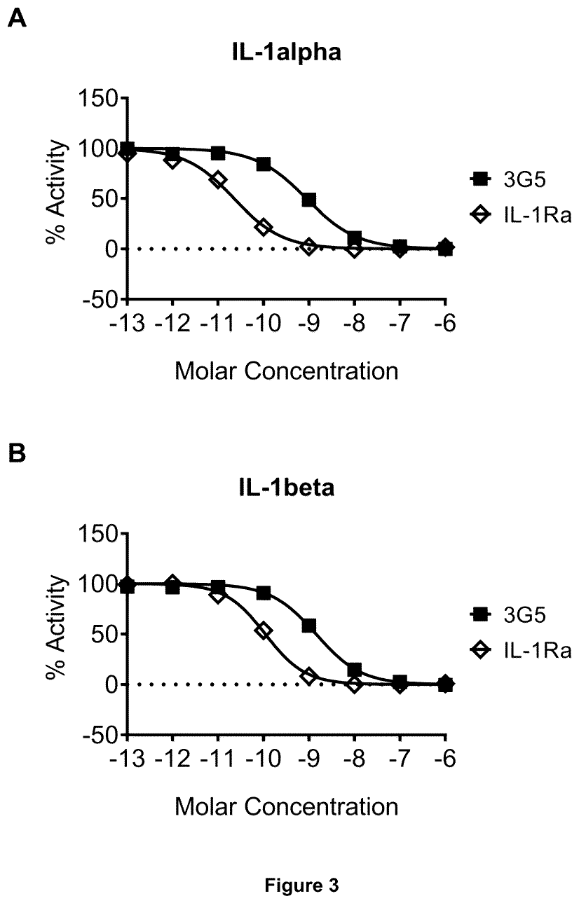

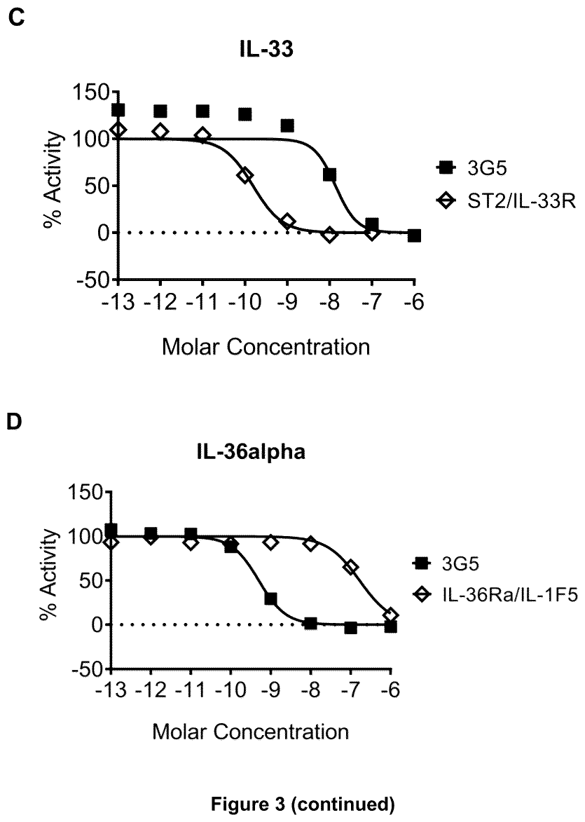

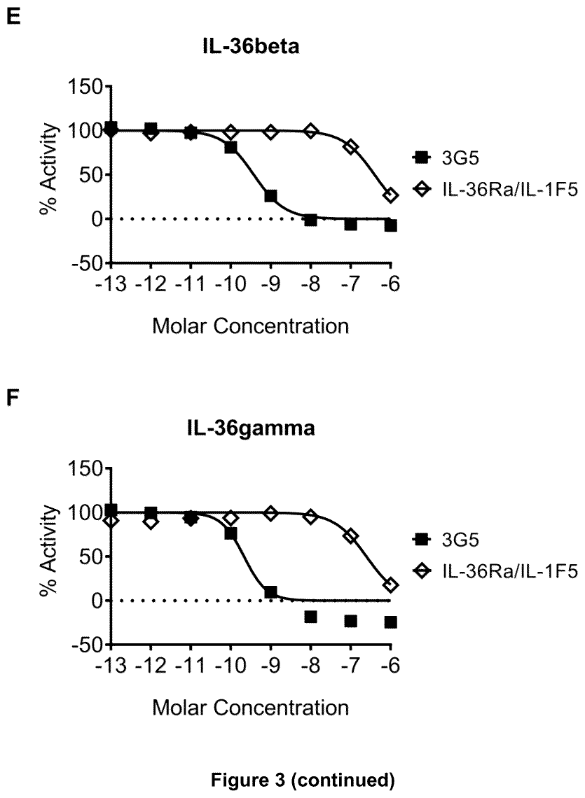

For analysis of IL-1 and IL-33 inhibition, HEK-Blue™ (IL-33/IL-1) cells were used as provided. For IL-36 analysis, HEK-Blue™ clones with stable expression of the IL-36 receptor (generated in house) were used. Briefly, HEK-Blue™ cells were seeded in 384-well plates and allowed to settle for a minimum of 2 hours. Cells were subsequently exposed to 43F5, 48A5, 3G5, natural antagonists, or isotype controls (as indicated in FIGS. 2 and 3) and incubated for 1 hour at 37° C., 5% CO₂, before addition of cytokines.

The cytokines were added in concentrations representing EC80 for the respective cytokine and batch: 0.2 ng/ml for IL-1α, 0.1-2 ng/ml for IL-1β, 0.2 ng/ml for IL-33, 0.02-1 ng/ml for IL-36α, 0.014-3 ng/ml for IL-36β and 0.005-1 ng/ml for IL-36γ. Cells were cultured for 16-18 hours at 37° C., 5% CO₂ and analyzed for activation of NF-κB and subsequent production of SEAP using QUANTI-Blue™ Solution measured at 620 nm using the SpectraMax i3x spectrophotometer.

### Affinity Measurements (Octet)

Protein A Biosensors were hydrated in blocking buffer for 10 min at room temperature before coating. The biosensors were coated with chimeric 3G5 by dipping the biosensors in 200 μl of antibody solution (10 pg/ml in blocking buffer) for 30 min at room temperature. The sensors were then incubated in 200 μl blocking buffer for 10 min before use.

kₐ and k_{d} were determined for four different concentrations of human IL1 RAP ectodomain (aa21-367) in blocking buffer, and K_{D} was then calculated by global fitting model (BLItz Pro version: 1.3.1.3). The four concentrations of human IL1RAP ectodomain (aa21-367) were 4 nM (0.16 μg/ml), 20 nM (0.8 μg/ml), 100 nM (4 μg/ml) and 500 nM (20 μg/ml).

The baseline was created in blocking buffer with a duration time of 30 s. Association was performed for 120 s in IL1RAP extracellular domain followed by 120 s dissociation in blocking buffer.

### Domain Mapping

The IL1RAP ectodomain consists of three domains (domain 1, domain 2, domain 3). To understand which of the three IL1RAP domains 43F5, 48A5 and 3G5 bind, a series of IL1RAP constructs were generated corresponding to the different domains, and binding to these was evaluated in an indirect ELISA assay.

Constructs were created using the amino acid sequence for IL1RAP given in Uniprot, ID Q9NPH3. Microtiter plates were coated with 100 ng of recombinant ectodomain hIL1RAP ‘Domain1+2+3’ (comprising domain 1, domain 2 and domain 3; aa21-367; positive control), recombinant hIL1RAP ‘Domain1+2’ (comprising domain 1 and domain 2; aa21-234), or recombinant hIL1RAP ‘Domain3’ (aa235-367) (100 μl/well) diluted in 0.01 M PBS, pH 7.4, and incubated overnight at 4° C. Plates were washed with ELISA washing buffer (0.01 M PBS, 0.05% Tween 20, pH 7.4) followed by a blocking step using 150 βl/well of ELISA blocking solution (PBS, 0.5% BSA, 0.05% Tween 20, pH 7.4). After 1 hour incubation at room temperature on agitation, the plates were washed again using ELISA washing buffer. A dilution series of antibodies 43F5, 48A5 and 3G5 in ELISA blocking solution was prepared individually (ranging with 4× dilution from 0.31 to 5000 ng/ml) and then transferred to the wells of the washed plates at 100 βl/well. Plates were incubated at room temperature for 1 hour on agitation and then washed with ELISA washing solution. One hundred βl/well of goat anti-human IgG conjugated to alkaline phosphatase was added and incubated for 1 hour at room temperature on agitation. The plates were washed using ELISA washing solution followed by addition of substrate (4-nitrophenyl phosphate disodium salt hexahydrate, Sigma-Aldrich, 1 mg/ml), 100 βl/well. The plates were thereafter incubated at room temperature on agitation and absorbance at 405 nm was measured consecutively for 30 min. Absorbance at 0 min was taken as background signal.

### Cross-Reactivity

Microtiter plates were coated with 100 ng of recombinant IL1RAP protein from different species (human, mouse, rat, cyno, rabbit, dog, pig) (100 βl/well) diluted in coating buffer (0.01 M PBS, pH 7.4), and incubated overnight at 4° C. The protein orthologs were coated in duplicates for each sample antibody. Plates were washed with ELISA washing buffer (0.01 M PBS, 0.05% Tween 20, pH 7.4) followed by a blocking step using 150 βl/well of ELISA blocking solution (PBS, 0.5% BSA, 0.05% Tween 20, pH 7.4). After 1 hour incubation at room temperature on agitation, the plates were washed with ELISA washing buffer. Antibodies 43F5, 48A5 and 3G5 were diluted in ELISA blocking solution to 1 μg/ml and transferred to the wells at 100 βl/well, in duplicate for each ortholog. Plates were incubated at room temperature for 1 hour on agitation and then washed with ELISA washing solution. One hundred βl/well of goat anti-human IgG conjugated to alkaline phosphatase was added and incubated for 1 hour at room temperature on agitation. The plates were washed using ELISA washing solution followed by addition of substrate (4-4-nitrophenyl phosphate disodium salt hexahydrate, Sigma-Aldrich, 1 mg/ml), 100 βl/well. The plates were thereafter incubated at room temperature on agitation and absorbance at 405 nm was measured consecutively for 30 min. Absorbance at 0 min was taken as background signal.

### Results

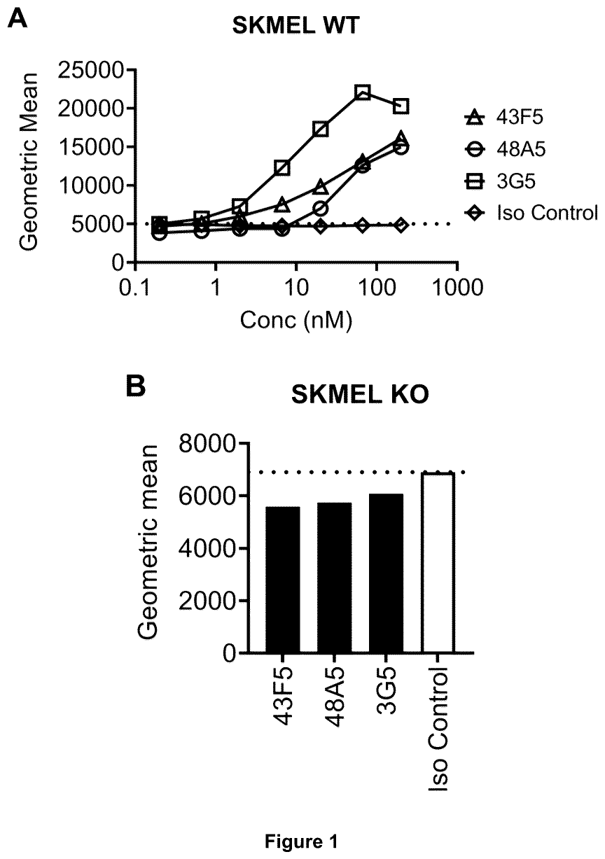

Antibody binding to IL1RAP-expressing cells Flow cytometry analysis of IL1RAP-expressing SKMEL-5 cells stained with 43F5, 48A5, 3G5 or isotype control antibody revealed a higher mean fluorescence intensity (MFI) for 43F5, 48A5 and 3G5 compared to the isotype control antibody (FIG. 1A), indicating binding of 43F5, 48A5 and 3G5 to the SKMEL-5 cells. No binding of 43F5, 48A5 and 3G5 was observed to SKMEL IL1RAP KO. (FIG. 1B).

### Inhibition of Cytokine Signaling

FIGS. 2 and 3 show the inhibitory activity of 43F5, 48A5 and 3G5 on IL-1α, IL-1β, IL-33, IL-36α, IL-36β and IL-36γ signaling in HEK cells. FIG. 2 shows optical density (OD) values at 620 nm with a high OD representing signaling downstream of the cytokine receptor. The antibodies 43F5, 48A5 and 3G5 induced a pronounced inhibition of IL-1α, IL-1β, IL-33, IL-36α, IL-36β and IL-36γ signaling, up to complete inhibition (blocking) of the signaling of these cytokines. FIG. 3 shows percent activity of signaling downstream of the cytokine receptor for the 3G5 antibody. The IC50 values for 3G5 are shown in Table 2.

TABLE 2
IC50 values [M] of 3G5 on the different signaling pathways, obtained from the
HEK-Blue ™ assay

| Antibody | IL-1α | IL-1β | IL-33 | IL-36α | IL-36β | IL-36γ |
|---|---|---|---|---|---|---|
| 3G5 | 8.780e−010 | 1.429e−009 | 1.375e−008 | 5.024e−010 | 3.760e−010 | 2.149e-010 |

### Affinity Measurements

Affinity measurement results are shown in Table 3 for 3G5, kₐ is the association rate constant for characterization of the antibody binding to the target, k_{d} is the dissociation rate constant for characterization of the antibody dissociation from the target, and K_{D} is the equilibrium dissociation constant between the antibody and its antigen, the ratio of k_{d}/kₐ. K_{D} corresponds to the antibody concentration at which 50% of the antigen binding sites are occupied at equilibrium. The lower the K_{D} value (lower concentration), the higher the affinity.

3G5 displayed affinity to human (h) IL1RAP in the nM range.

TABLE 3
Affinity measurements for 3G5

| | K_{D} | Kₐ | K_{d} |
|---|---|---|---|
| Antibody | (nM) | (1/Ms) | (1/s) |
| 3G5 | 0.2853 | 9.883E4 | 2.82E−5 |

### Domain Mapping

The results from the domain mapping are summarized in Table 4. The control polyclonal antibody KMT-2 bound to all domains, whereas 43F5, 48A5 and 3G5 only bound to IL1RAP constructs containing domain 3, suggesting that these antibodies all bind domain 3 of IL1RAP.

TABLE 4
Domain mapping for 43F5, 48A5 and 3G5

| | Domain1 + | | | |
|---|---|---|---|---|
| | 2 + 3 | Domain1 + 2 | Domain3 | Suggested |
| Antibody | (aa21-367) | (aa21-234) | (aa235-367) | epitope |
| 43F5 | + | − | + | Domain 3 |
| 48A5 | + | − | + | Domain 3 |
| 3G5 | + | − | + | Domain 3 |
| KMT-2 | + | + | + | Polyclonal |

### Cross-Reactivity

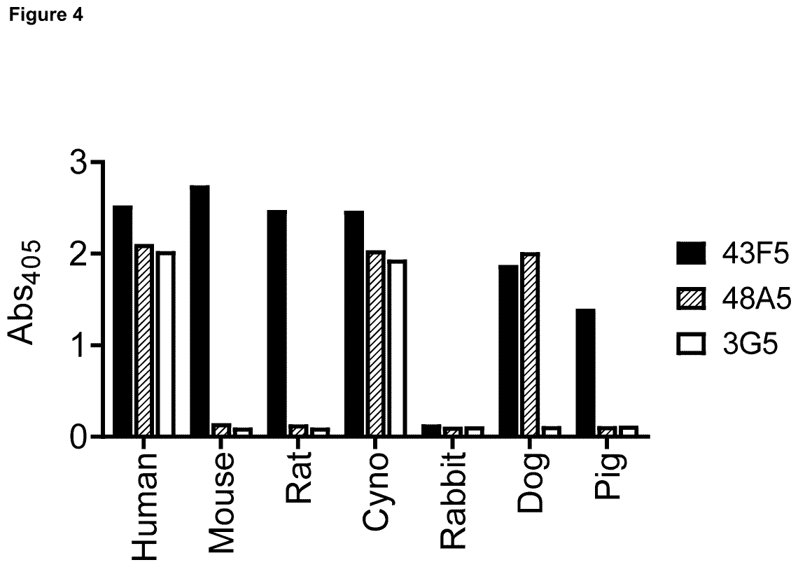

FIG. 4 and Table 5 show the cross-reactivity of 43F5, 48A5 and 3G5 to IL1 RAP from different species. 43F5 is cross-reactive to IL1 RAP from human, mouse, rat, cynomolgus monkey (cyno), dog and pig, but has no cross-reactivity to IL1 RAP from rabbit. 48A5 is cross-reactive to IL1RAP from human, cynomolgus monkey (cyno) and dog, but has no cross-reactivity to IL1 RAP from mouse, rat, rabbit or pig. 3G5 is cross reactive to IL1 RAP from human and cynomolgus monkey (cyno), but has no cross-reactivity to IL1 RAP from mouse, rat, rabbit, dog or pig.

TABLE 5
Cross-reactivity of 43F5, 48A5 and 3G5

| Anti- | IL1RAP | IL1RAP | IL1RAP | IL1RAP | IL1RAP | IL1RAP | IL1RAP |
|---|---|---|---|---|---|---|---|
| body | (human) | (mouse) | (rat) | (cyno) | (rabbit) | (dog) | (pig) |
| 43F5 | + | + | + | + | − | + | + |
| 48A5 | + | − | − | + | − | + | − |
| 3G5 | + | − | − | + | − | − | − |

### Conclusions

43F5, 48A5 and 3G5 all selectively bind IL1 RAP on the cell membrane and completely inhibit IL-1α, IL-1β, IL-33, IL-36α, IL-36β, and IL-36γ signaling. They all bind to domain 3 of IL1 RAP and bind human and cynomolgus monkey IL1 RAP.

### Example 3: Epitope Mapping of 43F5, 48A5 and 3G5

### Aim

This example illustrates the epitope mapping for 43F5, 48A5 and 3G5, i.e. the process of identifying the epitope on IL1 RAP to which these antibodies bind.

Material and methods

### ELISA

Based on the domain mapping (Example 2), chimeric constructs of murine/human IL1RAP were created, where human sequences were grafted onto murine IL1RAP: Chimeric IL1RAP.H3 (H3 corresponding to aa239-251), IL1RAP.H4 (H4 corresponding to aa263-277), IL1RAP.H5 (H5 corresponding to aa292-306) and IL1RAP.H6 (H6 corresponding to aa323-343). Microtiter plates were coated with 100 ng of chimeric IL1RAP.H3, IL1RAP.H4, IL1RAP.H5 and IL1RAP.H6 (100 μl/well) diluted in coating buffer (0.01 M PBS, pH 7.4), and incubated overnight at 4° C. The proteins were coated in duplicate for each antibody sample. Plates were washed with ELISA washing buffer (0.01 M PBS, 0.05% Tween 20, pH 7.4) followed by a blocking step using 150 βl/well of ELISA blocking solution (PBS, 0.5% BSA, 0.05% Tween 20, pH 7.4). After 1 hour incubation at room temperature on agitation, the plates were washed using ELISA washing buffer. Antibodies 43F5, 48A5 and 3G5 were diluted in ELISA blocking solution to 1 μg/ml and were added to the wells at 100 βl/well in duplicate for each coated chimeric protein. Plates were incubated at room temperature for 1 hour on agitation and then washed with ELISA washing solution. 100 βl/well of goat anti-human IgG conjugated to alkaline phosphatase was added and incubated 1 hour at room temperature on agitation.

The plates were washed using ELISA washing solution followed by addition of substrate (4-nitrophenyl phosphate disodium salt hexahydrate, Sigma-Aldrich, 1 mg/ml), 100 βl/well. The plates were thereafter incubated at room temperature on agitation and absorbance at 405 nm was measured consecutively for 30 min. Absorbance at 0 min was taken as background signal.

### Results

The epitope mapping results using murine IL1RAP with the grafted human sequences for regions H3, H4, H5 and H6 are shown in Table 6. 43F5 binding to murine IL1RAP was restored when the human sequence for the H3 or the H5 region was grafted, 48A5 binding to murine IL1RAP was restored when the human sequence for the H4 region was grafted, and 3G5 binding to murine IL1RAP was restored when the human sequence for the H3 region was grafted. The positive control polyclonal antibody KMT-3 bound to all chimeric versions of IL1RAP.

TABLE 6
Epitope mapping of 43F5, 48A5 and 3G5 using chimeric murine/human IL1RAP

| | Chimeric | Chimeric | Chimeric | Chimeric | Suggested |
|---|---|---|---|---|---|
| Antibody | IL1RAP.H3 | IL1RAP.H4 | IL1RAP.H5 | IL1RAP.H6 | epitope |
| 43F5 | + | − | + | − | H3/H5 |
| 48A5 | − | (+) | − | − | Multiple/H4 |
| 3G5 | (+) | − | − | − | Multiple/H3 |
| KMT-3 | + | + | + | + | Polyclonal |

### Conclusions

43F5, 48A5 and 3G5 all bind distinct epitopes on IL1RAP; 43F5 interacts with the H3 and H5 region of human IL1RAP, 48A5 interacts with the H4 region, and 3G5 with the H3 region.

### Example 4: Binding of 3G5 to Human Monocytes

### Aim

This example illustrates the binding profile of 3G5 to the surface of CD14+ human monocytes.

### Material and Methods

Human blood (10 ml) was collected into EDTA-coated collection tubes and stored at room temperature. All cell preparation and washing procedures were performed cold using pre-cooled buffers and incubations at 4° C.: Cells were stained with 3G5 (in effector-function silent hIgG1-LALA format) or isotype control antibody (hIgG1-LALA) conjugated to Alexa488 at the indicated concentrations, and also with anti-CD14 PE/Dazzle 594, anti-CD16 PerCP-Cy5.5, anti-CD45 APC-A700 (all BioLegend), and the live/dead dye Aqua V-500 in 50 ul of whole blood for 30 min at 4° C. Red blood cells were lysed in ACK (Gibco) solution for 10 min at 4° C. The cells were subsequently washed in PBS-1% BSA, fixed with 1×CellFix (BD) and analyzed on CytoFlex (Beckman Coulter).

### Results

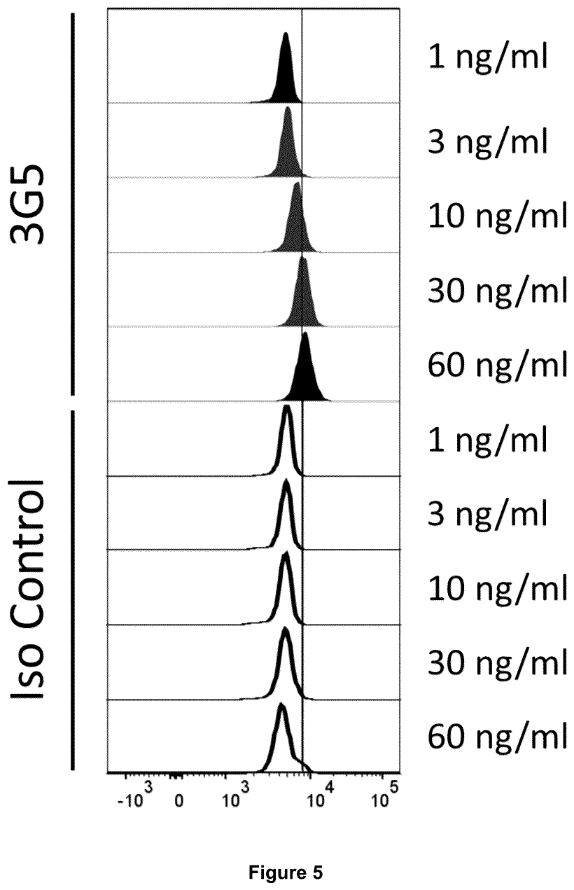

Flow cytometry analyses show dose-dependent staining of CD14+ human blood monocytes by Alexa488-conjugated 3G5 antibody, but not isotype control antibody (FIG. 5). This indicates that 3G5 binds to IL1RAP expressed on the surface of these cells.

### Conclusions

3G5 is capable of binding human CD14+ monocytes.

### Example 5: Inhibition of IL-33 Signaling in HUVEC by 3G5

### Aim

This example illustrates the inhibitory capacity of 3G5 on IL-6 and IL-8 release by HUVEC (human umbilical vein endothelial cells) in response to IL-33-stimulation.

### Material and Methods

HUVEC were cultured in F-12K media supplemented with Large Vessel Endothelial Supplement (ThermoFisher Scientific) and 100 U/ml penicillin/streptomycin. Cells were serum starved in a flat-bottom 96-well plate at 30,000 cells/well and incubated overnight. The following day, cells were pre-treated with 3G5 (in effector-function silent hIgG1-LALA format), isotype control antibody (hIgG1-LALA) or melrilimab (anti-IL-33R antibody) for 30 minutes at 20 μg/ml, 37° C., 5% CO₂ prior to stimulation with IL-33 at 30 ng/ml. Additional controls were included where no antibody was added to IL-33-stimulated HUVEC, or where neither antibody nor IL-33 were added (cells only). After 24 hours, supernatants were collected, and IL-6 and IL-8 ELISA performed.

### Results

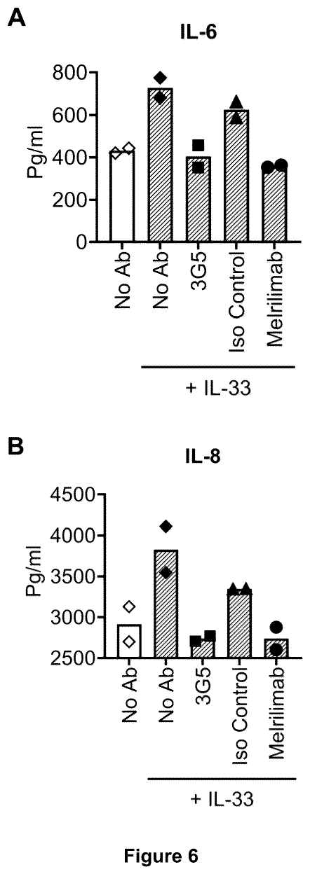

Blockade of IL-33 signaling by 3G5 resulted in reduced release of IL-6 (FIG. 6A) and IL-8 (FIG. 6B) by HUVEC in response to IL-33 stimulation. This effect was comparable to the anti-IL33R antibody melrilimab. No major effect was observed by isotype control antibody on IL-6 or IL-8 release.

### Conclusions

IL1RAP blockade by 3G5 impacts downstream effects of IL-1R signaling mediated by IL-33 such that the release of various pro-inflammatory cytokines and chemokines is reduced.

### Example 6: ADCC effect of 3G5 on SKMEL-5 melanoma cells

### Aim

This example illustrates the antibody-dependent cellular cytotoxicity (ADCC) effect of 3G5 expressed either in effector-function silent hIgG1-LALA format (LALA), or hIgG1-wild type (WT) format.

### Material and Methods

The malignant melanoma cell line SKMEL-5, expressing IL1RAP on the cell surface, were used as target cells in an in vitro ADCC assay. Target cells were seeded in a 96-well plate at a density of 10,000 cells per well. Subsequently, 3G5-WT, 3G5-LALA or isotype control antibodies were added to the wells at 100, 10 or 0 ng/ml and incubated for 30 min before 100,000 effector NK cells were added to each well. The NK cells were extracted from Leukocyte Concentrates by using an NK cell negative cell isolation kit according to manufacturer's instructions (Miltenyi Biotech). Non-specific hIgG1-WT and hIgG1-LALA antibodies were used as isotype control antibodies. The degree of cell death was assessed by detection of DAPI positive cells using a FACS LSR Fortessa flow cytometer (BD) after 18 hours in culture.

### Results

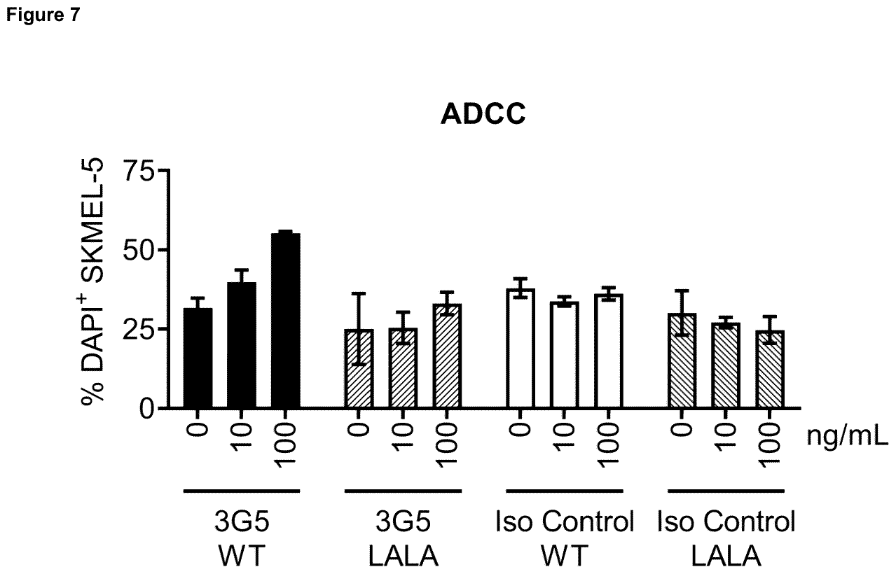

3G5 directs NK cells to kill IL1RAP-expressing SKMEL-5 cells by ADCC in an antibody-specific, dose-dependent manner (FIG. 7). No such effect was observed by 3G5 expressed in an effector-function silent hIgG1-LALA format, or by any of the isotype control antibodies.

### Conclusions

IL1RAP binding by 3G5 induces ADCC of IL1RAP-expressing SKMEL-5 cells.

Sequences
3G5 CDR sequences (defined according to IMGT)
Variable light chain complementarity-
determining region 1 (CDR-L1)
SEQ ID NO: 1
ENIYSN
Variable light chain complementarity-determining
region 2 (CDR-L2)
SEQ ID NO: 2
AA
Variable light chain complementarity-determining
region 3 (CDR-L3)
SEQ ID NO: 3
QHFWTTPRT
Variable heavy chain complementarity-determining
region 1 (CDR-H1)
SEQ ID NO: 4
GFTFSDYA
Variable heavy chain complementarity-determining
region 2 (CDR-H2)
SEQ ID NO: 5
ITDGGSYA
Variable heavy chain complementarity-determining
region 3 (CDR-H3)
SEQ ID NO: 6
SRDRWPYYFDF
43F5 CDR sequences (defined according to IMGT)
Variable light chain complementarity-determining
region 1 (CDR-L1)
SEQ ID NO: 7
QSIDSY
Variable light chain complementarity-determining
region 2 (CDR-L2)
SEQ ID NO: 8
GA
Variable light chain complementarity-determining
region 3 (CDR-L3)
SEQ ID NO: 9
QCTDYSINYIGA
Variable heavy chain complementarity-determining
region 1 (CDR-H1)
SEQ ID NO: 10
GFSLSNYW
Variable heavy chain complementarity-determining
region 2 (CDR-H2)
SEQ ID NO: 11
ITSSDNT
Variable heavy chain complementarity-determining
region 3 (CDR-H3)
SEQ ID NO: 12
ARDISGINSVVL
48A5 CDR sequences (defined according to IMGT)
Variable light chain complementarity-determining
region 1 (CDR-L1)
SEQ ID NO: 13
QSVYNDAY
Variable light chain complementarity-determining
region 2 (CDR-L2)
SEQ ID NO: 14
LA
Variable light chain complementarity-determining
region 3 (CDR-L3)
SEQ ID NO: 15
QGAYYSNDWYYV
Variable heavy chain complementarity-determining
region 1 (CDR-H1)
SEQ ID NO: 16
GFSLSYD
Variable heavy chain complementarity-determining
region 2 (CDR-H2)
SEQ ID NO: 17
IDTSSNT
Variable heavy chain complementarity-determining
region 3 (CDR-H3)
SEQ ID NO: 18
ARGGVHAYAYAPAAFDP
Variable chain regions as in 3G5 antibody
Light chain variable region
SEQ ID NO: 19
DIQMTQSPTSLSVSVGETVTITCRISENIYSNLAWYQQKEGKSPQLLVYA
ATKLADGVPSRFSGSGSGTQYSLKINSLQSEDFGNYYCQHFWTTPRTFGG
GTKVEIK
Heavy chain variable region
SEQ ID NO: 20
EVQLVESGGVLVKPGGSLKLSCAASGFTFSDYAMSWVRQTPEKRLEWVAT
ITDGGSYAYYTDDVKGRFTVSRDNARNNLFLQMSHLRSEDTGIYYCSRDR
WPYYFDFWGRGTTLTVSS
Variable chain regions as in 43F5 antibody
Light chain variable region
SEQ ID NO: 21
DVVMTQTPASVSEPVGGTVTINCQASQSIDSYLSWYQQKPGQPPKFLIYG
ASNLASGVPSRFKGSRSGTEYTLTISDLECADAATYYCQCTDYSINYIGA
FGGGTKVVVE
Heavy chain variable region
SEQ ID NO: 22
QSLEESGGRLVTPGTPLTLTCKASGFSLSNYWMSWVRQAPGKGLEWIGFI
TSSDNTYYASWAKGRFTISKASSTTVGLKMTSPTTEDTATYFCARDISGI
NSVVLWGQGTLVTVSS
Variable chain regions as in 48A5 antibody
Light chain variable region
SEQ ID NO: 23
APVLTQTPSPVSAAVGGTVTISCQSSQSVYNDAYLAWYQQKPGQPPKLLI
YLASTLASGVPSRFSGSGSGTQFTLTISGVQCEDAATYYCQGAYYSNDWY
YVFGGGTEVVVK
Heavy chain variable region
SEQ ID NO: 24
QEQLEESEGGLFKPTETLTLTCTVSGFSLSYDITWVRQAPGNGLEWIGTI
DTSSNTYHATWAKSRSTITKNTNLNTVTLKMTSLTAADTATYFCARGGVH
AYAYAPAAFDPWGPGTLVTISS
Constant regions
Immunoglobulin kappa constant light chain (light
chain constant region) (Km3 allotype)
SEQ ID NO: 35
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SENRGEC
Bold and underlined amino-acid residues indicate
the residues that are altered when the
LALA-mutation is introduced.
Immunoglobulin IgG1 constant heavy chain (heavy
chain constant region) (za allotype)
SEQ ID NO: 36
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
Immunoglobulin IgG1 constant heavy chain (heavy
chain constant region) (za allotype) with
‘LALA’ mutation
SEQ ID NO: 37
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG
Human IL1RAP
Full-length human IL1RAP
SEQ ID NO: 38
MTLLWCVVSLYFYGILQSDASERCDDWGLDTMRQIQVFEDEPARIKCPLF
EHFLKFNYSTAHSAGLTLIWYWTRQDRDLEEPINFRLPENRISKEKDVLW
FRPTLLNDTGNYTCMLRNTTYCSKVAFPLEVVQKDSCFNSPMKLPVHKLY
IEYGIQRITCPNVDGYFPSSVKPTITWYMGCYKIQNFNNVIPEGMNLSFL
IALISNNGNYTCVVTYPENGRTFHLTRTLTVKVVGSPKNAVPPVIHSPND
HVVYEKEPGEELLIPCTVYFSFLMDSRNEVWWTIDGKKPDDITIDVTINE
SISHSRTEDETRTQILSIKKVTSEDLKRSYVCHARSAKGEVAKAAKVKQK
GNRCGQ
Domain 3 of IL1RAP
SEQ ID NO: 39
PPVIHSPNDHVVYEKEPGEELLIPCTVYFSFLMDSRNEVWWTIDGKKPDD
ITIDVTINESISHSRTEDETRTQILSIKKVTSEDLKRSYVCHARSAKGEV
AKAAKVK
H3 region of human IL1RAP
SEQ ID NO: 40
NAVPPVIHSPNDH
H4 region of human IL1RAP
SEQ ID NO: 41
LIPCTVYFSFLMDSR
H5 region of human IL1RAP
SEQ ID NO: 42
ITIDVTINESISHSR
H6 region of human IL1RAP
SEQ ID NO: 43
SEDLKRSYVCHARSAKGEVAK

1. An antibody or antigen-binding fragment thereof, wherein the antibody or antigen-binding fragment comprises:
a light chain variable region comprising
a) a CDR-L1 comprising or consisting of an amino acid sequence selected from the group consisting of ENIYSN (SEQ ID NO: 1), QSIDSY (SEQ ID NO: 7) and QSVYNDAY (SEQ ID NO: 13);
b) a CDR-L2 comprising or consisting of an amino acid sequence selected from the group consisting of AA (SEQ ID NO: 2), GA (SEQ ID NO: 8) and LA (SEQ ID NO: 14); and
c) a CDR-L3 comprising or consisting of an amino acid sequence selected from the group consisting of QHFWTTPRT (SEQ ID NO: 3), QCTDYSINYIGA (SEQ ID NO: 9) and QGAYYSNDWYYV (SEQ ID NO: 15);
and/or
a heavy chain variable region comprising a CDR-H1 comprising or consisting of an amino acid sequence selected from the group consisting of GFTFSDYA (SEQ ID NO: 4), GFSLSNYW (SEQ ID NO: 10) and GFSLSYD (SEQ ID NO: 16);
d) a CDR-H2 comprising or consisting of an amino acid sequence selected from the group consisting of ITDGGSYA (SEQ ID NO: 5), ITSSDNT (SEQ ID NO: 11) and IDTSSNT (SEQ ID NO: 17); and
e) a CDR-H3 comprising or consisting of an amino acid sequence selected from the group consisting of SRDRWPYYFDF (SEQ ID NO: 6), ARDISGINSVVL (SEQ ID NO: 12) and ARGGVHAYAYAPAAFDP (SEQ ID NO: 18).

2. The antibody or antigen-binding fragment thereof according to claim 1, wherein the antibody or antigen-binding fragment thereof comprises a light chain variable region comprising or consisting of the amino acid sequence selected from the group consisting of SEQ ID NOs: 19, 21 and 23; or an amino acid sequence having at least 70% sequence identity thereto, for example at least 80%, 85%, 90%, 95%, 98% or 99% sequence identity.

3. The antibody or antigen-binding fragment thereof according to claim 1, wherein the antibody or antigen-binding fragment thereof comprises a heavy chain variable region comprising or consisting of the amino acid sequence selected from the group consisting of SEQ ID NOs: 20, 22 and 24; or an amino acid sequence having at least 70% sequence identity thereto, for example at least 80%, 85%, 90%, 95%, 98% or 99% sequence identity.

4. The antibody or antigen-binding fragment thereof according to claim 1, wherein the antibody or antigen-binding fragment thereof comprises:
a) a light chain constant region, or part thereof, such as a kappa or lambda light chain; and/or
b) a heavy chain constant region, or part thereof, such as an immunoglobulin subclass selected from the group consisting of IgG1, IgG2, IgG3 and IgG4.

5. The antibody or antigen-binding fragment thereof according to claim 1, wherein the antibody or antigen-binding fragment thereof comprises an Fc region, optionally wherein the Fc region is mutated and/or non-naturally occurring.

6. The antibody or antigen-binding fragment thereof according to claim 1 comprising or consisting of an intact antibody.

7. The antibody or antigen-binding fragment thereof according to claim 1 comprising or consisting of an antigen-binding fragment selected from the group consisting of Fv fragments (e.g. single chain Fv and disulphide-bonded Fv), Fab-like fragments (e.g. Fab fragments, Fab′ fragments and F(ab)₂ fragments) and domain antibodies (e.g. single V_{H} variable domains or V_{L} variable domains).

8. The antibody or antigen-binding fragment thereof according to claim 1, wherein the antibody or antigen-binding fragment thereof is capable of inhibiting signaling of interleukin-1 (IL-1) family cytokine ligands and/or receptors.

9. The antibody or antigen-binding fragment thereof according to claim 1, wherein the antibody or antigen-binding fragment thereof is capable of inhibiting signaling of at least one cytokine selected from the group consisting of IL-1α, IL-1β, IL-33, IL-36α, IL-36β and IL-36γ, or any combination thereof.

10. The antibody or antigen-binding fragment thereof according to claim 1, wherein the antibody or antigen-binding fragment exhibits one or more of the following properties:
a) a binding affinity (K_{D}) for IL1RAP characterized by a K_{D}-value of 3 nM or less;
b) binding to domain 3 of IL1RAP;
c) binding to aa239 to 251, 263 to 277, 292 to 306, and/or 323 to 343 of IL1RAP;
d) cross-reactivity with IL1RAP from mouse, rat, cynomolgus monkey, dog and/or pig;
e) an inhibitory action on IL-1α signaling;
f) an inhibitory action on IL-1β signaling;
g) an inhibitory action on IL-33 signaling;
h) an inhibitory action on IL-36α signaling;
i) an inhibitory action on IL-36β signaling;
j) an inhibitory action on IL-36γ signaling.

11. The antibody or antigen-binding fragment thereof according to claim 1, wherein the antibody or antigen-binding fragment is capable of inducing ADCC of cells expressing IL1RAP.

12. The antibody or antigen-binding fragment thereof according to claim 1, wherein the antibody or antigen-binding fragment is not capable of inducing ADCC of cells expressing IL1RAP.

13. A polynucleotide encoding an antibody or antigen-binding fragment thereof according to claim 1 or a component polypeptide chain thereof.

14. A vector comprising a polynucleotide according to claim 13.

15. A recombinant host cell comprising a polynucleotide according to claim 13.

16. A method for producing an antibody or antigen-binding fragment according to claim 1, the method comprising culturing a recombinant host cell comprising a polynucleotide encoding an antibody or antigen-binding fragment thereof according to claim 1 or a component polypeptide chain thereof, under conditions which permit expression of the encoded antibody or antigen-binding fragment thereof.

17. A pharmaceutical composition comprising the antibody or antigen-binding fragment thereof according to claim 1, in a pharmaceutical composition, wherein the composition further comprises a pharmaceutically-acceptable diluent, carrier or excipient.

18. An antibody or antigen-binding fragment thereof according to claim 1, for use in medicine.

19. An antibody or antigen-binding fragment thereof according to claim 1, for use in the prevention, treatment, alleviation, detection and/or diagnosis of a disease or disorder susceptible to treatment with an inhibitor of IL-1α, IL-1β, IL-33, IL-36α, IL-36β and/or IL-36γsignaling, optionally wherein the disease or disorder is associated with cells expressing IL1RAP.

20. The antibody or the antigen-binding fragment thereof for use, according to claim 19, wherein the disease or disorder is an inflammatory and/or fibrotic disease or disorder.

21. An antibody or antigen-binding fragment thereof according to claim 1, for use in inducing cell death and/or inhibiting the growth and/or proliferation of pathological cells associated with a neoplastic disorder in a subject, or stem cells or progenitor cells thereof, wherein the cells express IL1RAP.

22. Use of an antibody or antigen-binding fragment thereof according to claim 1, in the preparation of a medicament for the prevention, treatment, alleviation, detection and/or diagnosis of a disease or disorder susceptible to treatment with an inhibitor of IL-1α, IL-1β, IL-33, IL-36α, IL-36β and/or IL-36γ signaling, optionally wherein the disease or disorder is associated with cells expressing IL1RAP.

23. Use of an antibody or antigen-binding fragment thereof according to claim 1, in the preparation of a medicament for the detection and/or diagnosis of a disease or disorder associated with cells expressing IL1RAP.

24. A method for the prevention and/or treatment and/or alleviation and/or detection and/or diagnosis of a disease or disorder susceptible to treatment with an inhibitor of IL-1α, IL-1β, IL-33, IL-36α, IL-36β and/or IL-36γ signaling in a subject, optionally wherein the disease or disorder is associated with cells expressing IL1RAP, comprising the step of administering to the subject an effective amount of an antibody or antigen-binding fragment thereof according to claim 1.

25. An in vitro method for the detection of cells expressing IL1RAP in a subject, the method comprising:
(a) providing a sample of cells from a subject to be tested, such as biopsy tissue or blood sample;
(b) optionally, extracting and/or purifying the cells present in the sample;
(c) contacting an antibody or antigen-binding fragment thereof according to claim 1 with cells present in the sample;
(d) determining whether the antibody or antigen-binding fragment thereof binds to the cells;
wherein the binding of the antibody or antigen-binding fragment thereof to the cells is indicative of the presence of a disease or disorder associated with cells expressing IL1RAP in the tissue of a subject.

26. An in vitro method for identifying a patient with a disease or disorder associated with cells expressing IL1RAP who would benefit from treatment with an antibody or antigen-binding fragment thereof according to claim 1, the method comprising:
(a) providing a sample of cells, such as biopsy tissue or blood sample from a patient to be tested;
(b) optionally, extracting and/or purifying the cells present in the sample;
(c) contacting an antibody or antigen-binding fragment thereof according to claim 1 with cells present in the sample; and
(d) determining whether the antibody or antigen-binding fragment thereof binds to the cells;
wherein the binding of the antibody or antigen-binding fragment thereof to cells expressing IL1RAP is indicative of a patient who would benefit from treatment with an antibody or antigen-binding fragment thereof according to claim 1.

27. A method for treating a patient with a disease or disorder associated with cells expression IL1RAP, the method comprising:
a) selecting a patient identified as having a disease or disorder associated with cells expressing IL1RAP according to claim 25; and
b) administering to said patient a therapeutic agent effective in the treatment of said disease or disorder.
